SEQUENCE LISTING (2 sequences)

SEQ ID NO: 1 - protein, 616 aa, Homo sapiens
MFRTAVMMAASLALTGAVVAHAYYLKHQFYPTVVYLTKSSPSMAVLYIQAFVLVFLLGKVMGKVFFGQLRAAEMEHLLERSWYAVTETCLAFTVFRDDFSPRFVALFTLLLFLKCFHWLAEDRVDFMERSPNISWLFHCRIVSLMFLLGILDFLFVSHAYHSILTRGASVQLVFGFEYAILMTMVLTIFIKYVLHSVDLQSENPWDNKAVYMLYTELFTGFIKVLLYMAFMTIMIKVHTFPLFAIRPMYLAMRQFKKAVTDAIMSRRAIRNMNTLYPDATPEELQAMDNVCIICREEMVTGAKRLPCNHIFHTSCLRSWFQRQQTCPTCRMDVLRASLPAQSPPPPEPADQGPPPAPHPPPLLPQPPNFPQGLLPPFPPGMFPLWPPMGPFPPVPPPPSSGEAVAPPSTSAALSRPSGAATTTAAGTSATAASATASGPGSGSAPEAGPAPGFPFPPPWMGMPLPPPFAFPPMPVPPAGFAGLTPEELRALEGHERQHLEARLQSLRNIHTLLDAAMLQINQYLTVLASLGPPRPATSVNSTEETATTVVAAASSTSIPSSEATTPTPGASPPAPEMERPPAPESVGTEEMPEDGEPDAAELRRRRLQKLESPVAH

SEQ ID NO: 2 - DNA, 3071 bp, Homo sapiens
  source 1..3071: mol_type = genomic DNA
gggggtggggagtgttgttaaccggaggggcagccgcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggagactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcctctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcttttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaaaaaaaaaa

(19) United States
(12) Patent Application Publication
(10) Pub. No.: US 2026/0131030 A1
(43) Pub. Date: May 14, 2026

(54) Compositions and Methods for Treating Disorders Associated with Cone Photoreceptor Cells

(71) Applicant: The Board of Regents of the University of Oklahoma, Norman, OK (US)

(72) Inventors: Xi-Qin DING, Edmond, OK (US); Fan YANG, St. Louis, MO (US); Sanford L. BOYE, Gainesville, FL (US)

(21) Appl. No.: 19/381,265

(22) Filed: Nov. 6, 2025

Related U.S. Application Data
(60) Provisional application No. 63/716,924, filed on Nov. 6, 2024.

(51) Int. Cl. A61K 48/00; A61K 9/00; A61K 38/45; A61P 27/02; C12N 15/86

(52) U.S. Cl. CPC A61K 48/0058; A61K 9/0048; A61K 38/45; A61K 48/0075; A61P 27/02; C12N 15/86; C12Y 203/02; C12N 2750/14143; C12N 2830/008

(57) ABSTRACT

A method of treating a cone photoreceptor cell-associated disorder in a subject in need of such therapy by administering to the eye of the subject a recombinant vector comprising: (a) a coding sequence encoding a synoviolin 1 (SYVN1) protein, and (b) a promoter region, wherein the promoter region is specific for cone photoreceptor cells, wherein the coding sequence is operatively linked to the promoter region, and wherein in vivo expression of the SYVN1 protein in the cone photoreceptor cells of the subject serves to treat the cone photoreceptor cell-associated disorder in the subject.

### CROSS REFERENCE TO RELATED APPLICATIONS

This application claims the benefit of U.S. Provisional Patent Application Ser. No. 63/716,924, filed Nov. 6, 2024, the content of which is hereby expressly incorporated herein by reference in its entirety.

### GOVERNMENT SUPPORT

This invention was made with government support under National Institutes of Health Grants EY027754 and EY033841. The government has certain rights in the invention.

### REFERENCE TO SEQUENCE LISTING SUBMITTED ELECTRONICALLY

The instant application contains, as a separate part of the present disclosure, a Sequence Listing which has been submitted via EFS-Web in computer readable form as an XML file. The Sequence Listing, created Nov. 5, 2025, is named “5864.177_Sequence_Listing.xml” and is 6,501 bytes in size. The entire contents of the Sequence Listing are hereby incorporated herein by reference.

### BACKGROUND

Photoreceptors are a specialized type of neuron found in the retina that are capable detecting light and converting that light signal into electrical signals. The two types of photoreceptors are “rod” photoreceptors, which are more sensitive to light and hence support vision in dim lighting, and “cone” photoreceptors, which are sensitive to specific wavelengths of light and hence support the perception of color, respond faster to stimuli than rods so perceive finer detail and more rapid changes in images than rods.

Cone photoreceptor cyclic nucleotide-gated (CNG) channels of cone photoreceptor cells are localized to the plasma membrane of photoreceptor outer segments and play an essential role in phototransduction and cellular Ca²⁺ homeostasis. In darkness, a portion of the channels are kept open by cyclic guanosine monophosphate (cGMP), maintaining a steady Na⁺ and Ca²⁺ influx. Light induces hydrolysis of cGMP by photoreceptor phosphodiesterase, resulting in the closure of the channels and hyperpolarization of the cone photoreceptor cell. Mutations in genes encoding the CNG channel subunits CNGA3 and CNGB3 are associated with achromatopsia, progressive cone dystrophy, and early-onset macular degeneration. These diseases are characterized by severely impaired daylight vision, lack of color discrimination, photophobia, and slow-progressing cone cell degeneration. There are over 100 and 40 disease-causing mutations in CNGA3 and CNGB3, respectively, accounting for about 80% of achromatopsia cases.

Loss of cone photoreceptor cells (also referred to herein as “cone cells” or “cones”) in patients with achromatopsia and cone dystrophy associated with CNG channel mutations has been documented by optical coherence tomography. Impaired cone function, progressive cone degeneration, and apoptotic cone death have also been characterized in mouse models of Cnga3- or Cngb3-deficiency. CNG channel-deficient mice display early-onset apoptotic cone death, with cone degeneration evident in the second postnatal week. Further studies demonstrate that cone death in CNG channel deficiency involves abnormally elevated cGMP/protein kinase G (PKG) signaling resulting from reduced cytosolic Ca²⁺ level, endoplasmic reticulum (ER) stress-associated apoptosis, dysregulation of cellular/ER Ca²⁺ homeostasis, and potentially impaired ER-associated degradation (ERAD). Retinas of CNG channel-deficient mice show elevated ER stress marker proteins, including Grp78/Bip, phospho-eukaryotic initiation factor 2α (phospho-eIF2α), and CCAAT/-enhancer-binding protein homologous protein (CHOP), upregulated cysteine protease calpains, and increased processing of caspase-12 and caspase-7. Expression/activity of the ER calcium channels, inositol 1,4,5-trisphosphate (IP₃R) receptor and ryanodine (RyR) receptor, are enhanced in the channel-deficient retina, and protein folding/trafficking is impaired, as evidenced by cone opsin mislocalization. Deletion of these channels by genetic/molecular approaches reduces ER stress/cone death and improves cone opsin outer segment localization. Treatment with molecular and chemical chaperones reduces ER stress/cone death in CNG channel deficiency. These findings support the fact that CNG channel-deficient cones suffer from impaired protein processing/ER proteostasis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that synoviolin 1 (SYVN1) is overexpressed in cones of CNG channel-deficient mice after intraocular injection with AAV5-IRBP/GNAT2-Syvn1. (A) Cnga3^{-/-} mice were injected with AAV5-IRBP/GNAT2-Syvn1 at P5 and analyzed at 4 months for retinal expression/cone localization of SYVN1 and PNA by immunofluorescence labeling. Shown are representative confocal images of SYVN1 and PNA labeling in the mouse retina. ONL, outer nuclear layer; INL, inner nuclear layer. (B) Cnga3^{-/-}/Nrl^{-/-} mice were injected at P5 with AAV5-IRBP/GNAT2-Syvn1 and analyzed at P40 for SYVN1 expression by immunoblotting. Shown are representative immunoblotting images and the corresponding quantitative analysis. Data are presented as mean±SEM of 3-4 assays using retinas prepared from 8-12 mice (***p<0.001).

FIG. 2 shows that treatment with AAV-Syvn1 increased cone survival in CNG channel-deficient mice. (A) Cnga3^{-/-} mice received AAV5-IRBP/GNAT2-Syvn1 at P5 and were analyzed at 4 months for cone by immunolabeling. Shown are representative confocal images of PNA labeling on retinal cross sections and corresponding quantitative analysis. Data are presented as mean±SEM (n=5-11 mice, ***p<0.001). (B) Cnga3^{-/-}/Nrl^{-/-} mice received AAV5-IRBP/GNAT2-Syvn1 at P5 and were analyzed at P40 for M-opsin expression by immunolabeling. Shown are representative confocal images of M-opsin labeling on retinal cross sections. ONL, outer nuclear layer; INL, inner nuclear layer.

FIG. 3 shows that treatment with AAV-Syvn1 reduced cone death in Cnga3^{-/-}/Nrl^{-/-} mice. Cnga3^{-/-}/Nrl^{-/-} mice received AAV5-IRBP/GNAT2-Syvn1 at P5 and were analyzed at P40 for cone death by TUNEL-labeling. Shown are representative confocal images of TUNEL-positive cells and the corresponding quantitative analysis. Data are presented as mean±SEM (n=6-10 mice, *p<0.05, **p<0.01).

FIG. 4 shows that treatment with AAV-Syvn1 increased outer segment localization of M-opsin in Cnga3^{-/-} mice. Cnga3^{-/-} mice received AAV5-IRBP/GNAT2-Syvn1 at P5 and were analyzed at 4 months for M-opsin localization by immunofluorescence labeling. Shown are representative confocal images of M-opsin labeling on retinal cross sections and the corresponding quantitative analysis. ONL, outer nuclear layer; INL, inner nuclear layer. OS, outer segment; Is, inner segment; OPL, outer plexiform layer. Data are presented as mean±SEM (n=5-8 mice, ***p<0.001).

FIG. 5 shows that treatment with AAV-Syvn1 reduced ER stress and ER stress downstream signaling in Cnga3^{-/-}/Nrl^{-/-} mice. Cnga3^{-/-}/Nrl^{-/-} mice received AAV5-IRBP/GNAT2-Syvn1 at P5 and were analyzed at P40 for expressions of (A) phospho-eIF2a and phospho-IRE1α, (B) CHOP, and (C) phospho-CREB and CREB313. Shown are representative immunoblotting images and the corresponding quantitative analysis. Data are presented as mean±SEM (n=8-12 mice, *p<0.05).

FIG. 6 shows that treatment with AAV-Syvn1 increased expression levels of ER retrotranslocation proteins in Cnga3^{-/-} Nrl^{-/-} mice. Cnga3^{-/-}/Nrl^{-/-} mice received AAV5-IRBP/GNAT2-Syvn1 at P5 and were analyzed at P40 for expressions of DERL1, SEL1L, and HERPUD1 by immunoblotting. Shown are representative immunoblotting images and the corresponding quantitative analysis. Data are presented as mean±SEM (n=8-12 mice, *p<0.05, **p<0.01).

FIG. 7 shows that treatment with AAV-Syvn1 enhanced the ubiquitin-proteasome activity in Cnga3^{-/-}/Nrl^{-/-} mice. Cnga3^{-/-}/Nrl^{-/-} mice received AAV5-IRBP/GNAT2-Syvn1 at P5 and were analyzed at P40 for expressions of (A) ubiquitin and (B) p53 by immunoblotting. Shown are representative immunoblotting images and the corresponding quantitative analysis. Data are presented as mean±SEM (n=8-12 mice, *p<0.05, **p<0.01).

FIG. 8 is a schematic which shows the role of ER retrotranslocation/ERAD in cone protection in CNG channel deficiency. In CNG channel deficiency, reduced cytosolic Ca²⁺ level stimulates ER Ca²⁺ channels, leading to increased Ca²⁺ release from the ER and a compensatory reduction of ER Ca²⁺ store/impaired ER Ca²⁺ homeostasis. Moreover, reduced cytosolic Ca²⁺ level also stimulates cGMP/PKG signaling, which is a strong inducer for the ER Ca²⁺ channels, impairing ER Ca²⁺ store. Impaired ER Ca²⁺ store in turn inhibits ER protein processing/protein folding, presumably via the regulation of the Ca²⁺-dependent ER chaperone proteins, leading to ER stress/cell death. Thus, suppressing ER Ca²⁺ channel activity improves protein trafficking in cones and reduces ER stress/cell death. The previous findings and results of the present work support that ER retrotranslocation/ERAD in cone photoreceptors is regulated at least by the following three factors: 1) ER Ca²⁺ store/homeostasis, 2) function of ER chaperones, and 3) expression levels of the retrotranslocon components. Increased Ca²⁺ store in the ER, enhanced protein folding/processing, and enhanced expression of the ER retrotranslocon components will facilitate ER retrotranslocation/ERAD and relieve ER stress, leading to preservation of photoreceptors.

The following abbreviations may be used herein:
BSA: bovine serum albumin;
CAMP: cyclic adenosine monophosphate;
cGMP: cyclic guanosine monophosphate;
CHOP: CCAAT-enhancer-binding protein homologous protein;
CNG: cyclic nucleotide-gated;
CREB: CAMP response element binding protein;
CREB313: CAMP responsive element binding protein 3 like 3;
DERL-1: degradation in ER protein 1;
DER3: Derlin-3, synonym for SYVN1;
ER: endoplasmic reticulum;
eIF2a: eukaryotic translation initiation factor 2 alpha;
ERAD: endoplasmic reticulum-associated degradation;
GRP75: the chaperone glucose-regulated protein 75;
HERPUD1: homocysteine inducible ER protein with ubiquitin like domain 1;
HRD1: synonym for SYVN1;
HRGP, human red/green pigment;
IP₃R1: inositol 1,4,5-trisphosphate receptor 1;
Ire1α: serine/threonine-protein kinase/endoribonuclease 1α;
P53: tumor protein p53;
PKG: cGMP/protein kinase G;
PNA: peanut agglutinin lectin;
RyR2: ryanodine receptor 2;
SEL1: ERAD E3 ligase adaptor subunit;
SYVN1: E3 ubiquitin-protein ligase synoviolin 1;
TBP: TATA binding protein;
TUDCA: tauroursodeoxycholic acid;
TUNEL: terminal deoxynucleotidyl transferase dUTP nick end-labeling;
Ub: ubiquitin;
UPR: unfolded protein response.

### DETAILED DESCRIPTION

The degeneration/death of cone photoreceptors will ultimately lead to loss of vision and blindness. One of the major pathological reasons for cone cell death is the accumulation of unfolded proteins in the ER which causes stress on the ER. Thus, the transfer of accumulated/unfolded proteins out of the ER for degradation is critical for the maintenance of ER homeostasis and cell viability. This process is known as ERAD. Using cone degeneration mouse models, it was discovered that overexpression of SYVN1, an ER retrotranslocation protein, reduces ER stress and preserves cone photoreceptor cells. The present disclosure is therefore directed to compositions and methods of treatment for preserving cone photoreceptor cells.

In one embodiment, the present disclosure is directed to a method of treating a cone photoreceptor cell-associated disorder in a subject in need of such therapy by administering to the eye of the subject a recombinant vector comprising: (a) a coding sequence encoding a synoviolin 1 (SYVN1) protein, and (b) a promoter region, wherein the promoter region is specific for cone photoreceptor cells, wherein the coding sequence is operatively linked to the promoter region, and wherein in vivo expression of the SYVN1 protein in the cone photoreceptor cells of the subject serves to treat the cone photoreceptor cell-associated disorder in the subject. The recombinant vector may further comprise an enhancer element upstream of the promoter, wherein the coding sequence is operatively linked to the enhancer element.

The SYVN1 protein encoded by the coding sequence may be a human SYVN1 protein, such as the protein having the amino acid sequence SEQ ID NO:1 (Table 1), or an SYVN1 protein orthologue having at least 90% identity to a human SYVN1 protein, such as a protein having at least 90% identity to SEQ ID NO:1. Alternatively (and/or in addition thereto), the coding sequence may comprise a sequence that is at least 90% identical to SEQ ID NO: 2 (Table 2). The cone photoreceptor cell-associated disorder may be selected from the group consisting of rod-cone dystrophy, cone-rod dystrophy, progressive cone dystrophy, retinitis pigmentosa (RP), Stargardt's Disease, Leber hereditary optic neuropathy (LHON), Leber congenital amaurosis (LCA), Best disease, adult vitelliform macular dystrophy (AVMD), X-linked retinoschisis, blindness, a color vision disorder, color blindness, blue cone monochromacy, achromatopsia, incomplete achromatopsia, protan defects, deutan defects, tritan defects, disorders of the central macula, dry age-related macular degeneration (dry AMD), wet age-related macular degeneration (wet AMD), geographic atrophy, retinal telangiectasia, macular telangiectasia, Coats' disease, diabetic retinopathy, retinal vein occlusions, retinal ischemia, Familial Exudative Vitreoretinopathy (FEVR), glaucoma, and Sorsby's fundus dystrophy.

The SYVN1 protein may be encoded by the human svyn1 coding sequence (HGNC No: 20738; NCBI gene: 84447; ENSG00000162298) or any SYVN1 coding sequence orthologue, particularly SYVN1 orthologues having at least 90% identity to human SYVN1 protein or the nucleic acid sequence encoding same. The SYVN1 protein having amino acid sequence SEQ ID NO:1 may be encoded by the nucleic acid coding sequences SEQ ID NO:2 (Table 2), or by any nucleic acid which encodes SEQ ID NO:1. The SYVN1 protein may be encoded by a coding sequence that is at least 90% identical to SEQ ID NO:2.

Examples of other mammalian SYVN1 coding sequence orthologues that can be inserted into the vector constructs used in the therapeutic methods of the present disclosure include, but are not limited, coding sequences designated by the following Ensembl gene identification numbers: ENSRBIG00000042217, ENSSBOG00000030991, ENSPPAG00000024469, ENSOGAG00000032940, ENSPTRG00000003868, ENSPCOG00000016867, ENSMLEG00000043824, ENSNLEG00000005509, ENSRROG00000039025, ENSGGOG00000002718, ENSPSMG00000000526, ENSANAG00000024672, ENSMMUG00000018781, ENSMICG00000016607, ENSPANG00000003694, ENSCCAG00000037953, ENSMNEG00000036252, ENSCATG00000000959, ENSPPYG00000003075, ENSCSAG00000007694, ENSCJAG00000002413, ENSMMMG00000009710, ENSUPAG00010009983, ENSCGRG00001009803, ENSODEG00000018215, ENSSVLG00005011924, ENSSVLG00005012005, ENSMAUG00000008845, ENSCPOG00000022104, ENSDORG00000003610, ENSJJAG00000011203, ENSCLAG00000017187, ENSMUSG00000024807, ENSHGLG00000011777, ENSPEMG00000004066, ENSMOCG00000014259, ENSOCUG00000022650, ENSRNOG00000020950, ENSSTOG00000003351, ENSMSIG00000030275, ENSNGAG00000010252, ENSBBBG00000007101, ENSUAMG00000012292, ENSNVIG00000017369, ENSCDRG00005010494, ENSDLEG00000003778, ENSBMSG00010009167, ENSFCAG00000030009, ENSCWAG00000013781, ENSBTAG00000005076, ENSCAFG00020014946, ENSCAFG00845025988, ENSTTRG00000017248, ENSEASG00005018208, ENSMPUG00000012218, ENSAMEG00000017667, ENSCHIG00000023946, ENSRFEG00010004691, ENSECAG00000022480, ENSBIXG00005002257, ENSPPRG00000024927, ENSPLOG00000019101, ENSPVAG00000007917, ENSMLUG00000023783, ENSMMNG00015021341, ENSSSCG00000039740, ENSUMAG00000008235, ENSVVUG00000027387, ENSOARG00020016284, ENSMMSG00000020141, ENSPCTG00005014381, ENSPTIG00000020574, ENSPSNG00000008474, ENSBMUG00000003047, and ENSCHYG00000003375.

TABLE 1
Human Synoviolin 1 Protein Sequence (SEQ ID NO: 1)

| | |
|---|---|
| 1 | mfrtavmmaa slaltgavva hayylkhqfy ptvvyltkss psmavlyiqa fvlvfllgkv |
| 61 | mgkvffgqlr aaemehller swyavtetcl aftvfrddfs prfvalftll lflkcfhwla |
| 121 | edrvdfmers pniswlfhcr ivslmfllgi ldflfvshay hsiltrgasv qlvfgfeyai |
| 181 | lmtmvltifi kyvlhsvdlq senpwdnkav ymlytelftg fikvllymaf mtimikvhtf |
| 241 | plfairpmyl amrqfkkavt daimsrrair nmntlypdat peelqamdnv ciicreemvt |
| 301 | gakrlpcnhi fhtsclrswf qrqqtcpter mdvlraslpa qsppppepad qgpppaphpp |
| 361 | pllpqppnfp qglippfppg mfplwppmgp fppvppppss geavappsts aalsrpsgaa |
| 421 | tttaagtsat aasatasgpg sgsapeagpa pgfpfpppwm gmplpppfaf ppmpvppagf |
| 481 | agltpeelra legherqhle arlqslrnih tlldaamlqi nqyltvlasl gpprpatsvn |
| 541 | steetattvv aaasstsips seattptpga sppapemerp papesvgtee mpedgepdaa |
| 601 | elrrrrlqkl espvah |

TABLE 2
Human Synoviolin 1 Coding Sequence (SEQ ID NO: 2)

| | |
|---|---|
| 1 | gggggtgggg agtgttgtta accggagggg cagccgcagt cgcgcggatt gagcgggctc |
| 61 | gcggcgctgg gttcctggtc tccgggccag ggcaatgttc cgcacggcag tgatgatggc |
| 121 | ggccagcctg gcgctgaccg gggctgtggt ggctcacgcc tactacctca aacaccagtt |
| 181 | ctaccccact gtggtgtacc tgaccaagtc cagccccagc atggcagtcc tgtacatcca |
| 241 | ggcctttgtc cttgtcttcc ttctgggcaa ggtgatgggc aaggtgttct ttgggcaact |
| 301 | gagggcagca gagatggagc accttctgga acgttcctgg tacgccgtca cagagacttg |
| 361 | tctggccttc accgtttttc gggatgactt cagcccccgc tttgttgcac tcttcactct |
| 421 | tcttctcttc ctcaaatgtt tccactggct ggctgaggac cgtgtggact ttatggaacg |
| 481 | cagccccaac atctcctggc tctttcactg ccgcattgtc tctcttatgt tcctcctggg |
| 541 | catcctggac ttcctcttcg tcagccacgc ctatcacagc atcctgaccc gtggggcctc |
| 601 | tgtgcagctg gtgtttggct ttgagtatgc catcctgatg acgatggtgc tcaccatctt |
| 661 | catcaagtat gtgctgcact ccgtggacct ccagagtgag aacccctggg acaacaaggc |
| 721 | tgtgtacatg ctctacacag agctgtttac aggcttcatc aaggttctgc tgtacatggc |
| 781 | cttcatgacc atcatgatca aggtgcacac cttcccactc tttgccatcc ggcccatgta |
| 841 | cctggccatg agacagttca agaaagctgt gacagatgcc atcatgtctc gccgagccat |
| 901 | ccgcaacatg aacaccctgt atccagatgc caccccagag gagctccagg caatggacaa |
| 961 | tgtctgcatc atctgccgag aagagatggt gactggtgcc aagagactgc cctgcaacca |
| 1021 | cattttccat accagctgcc tgcgctcctg gttccagcgg cagcagacct gccccacctg |
| 1081 | ccgtatggat gtccttcgtg catcgctgcc agcgcagtca ccaccacccc cggagcctgc |
| 1141 | ggatcagggg ccaccccctg ccccccaccc cccaccactc ttgcctcagc cccccaactt |
| 1201 | cccccagggc ctcctgcctc cttttcctcc aggcatgttc ccactgtggc cccccatggg |
| 1261 | cccctttcca cctgtcccgc ctccccccag ctcaggagag gctgtggctc ctccatccac |
| 1321 | cagtgcagcc ctttctcggc ccagtggagc agctacaacc acagctgctg gcaccagtgc |
| 1381 | tactgctgct tctgccacag catctggccc aggctctggc tctgccccag aggctggccc |
| 1441 | tgcccctggt ttccccttcc ctcctccctg gatgggtatg cccctgcctc caccctttgc |
| 1501 | cttcccccca atgcctgtgc cccctgcggg ctttgctggg ctgaccccag aggagctacg |
| 1561 | agctctggag ggccatgagc ggcagcacct ggaggcccgg ctgcagagcc tgcgtaacat |
| 1621 | ccacacactg ctggacgccg ccatgctgca gatcaaccag tacctcaccg tgctggcctc |
| 1681 | cttggggccc ccccggcctg ccacttcagt caactccact gaggagactg ccactacagt |
| 1741 | tgttgctgct gcctcctcca ccagcatccc tagctcagag gccacgaccc caaccccagg |
| 1801 | agcctcccca ccagcccctg aaatggaaag gcctccagct cctgagtcag tgggcacaga |
| 1861 | ggagatgcct gaggatggag agcccgatgc agcagagctc cgccggcgcc gcctgcagaa |
| 1921 | gctggagtct cctgttgccc actgacactg ccccagccca gccccagcct ctgctctttt |
| 1981 | gagcagccct cgctggaaca tgtcctgcca ccaagtgcca gctccctctc tgtctgcacc |
| 2041 | agggagtagt acccccagct ctgagaaaga ggcggcatcc cctaggccaa gtggaaagag |
| 2101 | gctggggttc ccatttgact ccagtcccag gcagccatgg ggatctcggg tcagttccag |
| 2161 | ccttcctctc caactcttca gccctgtgtt ctgctggggc catgaaggca gaaggtttag |
| 2221 | cctctgagaa gccctcttct tcccccaccc ctttccagga gaaggggctg cccctccaag |
| 2281 | ccctacttgt atgtgcggag tcacactgca gtgccgaaca gtattagctc ccgttcccaa |
| 2341 | gtgtggactc cagaggggct ggaggcaagc tatgaacttg ctcgctggcc cacccctaag |
| 2401 | actggtaccc atttcctttt cttaccctga tctccccaga agcctcttgt ggtggtggct |
| 2461 | gtgcccccta tgccctgtgg catttctgcg tcttactggc aaccacacaa ctcagggaaa |
| 2521 | ggaatgcctg ggagtggggg tgcaggcggg cagcactgag ggaccctgcc ccgcccctcc |
| 2581 | ccccaggccc ctttcctctg cagcttctca agtgagactg acctgtctca cccagcagcc |
| 2641 | actgcccagc cgcactccag gcaagggcca gtgcgcctgc tcctgaccac tgcaatccca |
| 2701 | gcgcccaagg aaggccactt ctcaactggc agaacttctg aagtttagaa ttggaattac |
| 2761 | ttccttacta gtgtcttttg gcttaaattt tgtcttttga agttgaatgc ttaatcccgg |
| 2821 | gaaagaggaa caggagtgcc agactcctgg tctttccagt ttagaaaagg ctctgtgcca |
| 2881 | aggagggacc acaggagctg ggacctgcct gcccctgtct tttccccttg gttttgtgtt |
| 2941 | acaagagttg ttggagacag tttcagatga ttatttaatt tgtaaatatt gtacaaattt |
| 3001 | taatagctta aattgtatat acagccaaat aaaaacttgc attaacaaaa aaaaaaaaaa |
| 3061 | aaaaaaaaaa a |

The present work investigated the effects of SYVN1 overexpression in cones in CNG channel deficiency. Overexpression of SYVN1 in cones was achieved by intraocular injection with AAV5-IRBP/GNAT2-Syvn1 (AAV-Syvn1). After treatment with AAV-Syvn1, CNG channel-deficient mice show increased cone density, reduced ER stress/cone death, enhanced expression levels of ER retrotranslocation proteins, and enhanced protein ubiquitination/proteasome degradation in the retina. These results demonstrate the important role of SYVN1/ERAD in cone preservation in CNG channel deficiency and shows how restoration of ER Ca²⁺ level/homeostasis reduces ER stress/cone death.

Following treatment, cone density in Cnga3^{-/-} mice was significantly increased, compared with untreated controls, outer segment localization of cone opsin was improved, and ER stress/apoptotic cell death was reduced. Overexpression of SYVN1 also led to increased expression levels of the retrotranslocon components, degradation in ER protein 1 (DERL1), ERAD E3 ligase adaptor subunit (SEL1L), and homocysteine inducible ER protein with ubiquitin like domain 1 (HERPUD1). Moreover, overexpression of SYVN1 enhanced protein ubiquitination/proteasome degradation in CNG channel-deficient retinas. It was found that manipulations that reduce ER stress/cone death, including resuming ER Ca²⁺ stores by deleting the ER calcium channels IP₃R1 or RyR2 or promoting protein folding in the ER by a chemical chaperone, are accompanied by increased expression of SYVN1 and other components of the ER retrotranslocon. This work demonstrates the role of SYVN1/ERAD in cone preservation in CNG channel deficiency and supports the strategy of promoting ERAD for cone protection.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, applications, published patent applications, and non-patent publications including published articles mentioned in the specification or referenced in any portion of this application, including but not limited to U.S. Provisional Patent Application Ser. No. 63/716,924, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists in the aspects of the present work. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of +20% or +10%, or +5%, or +1%, or +0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 75% of the time, or at least 80% of the time, or at least 85% of the time, or at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

Use of the word “we,” “us,” and/or “our” as a pronoun in the present disclosure refers generally to laboratory personnel, technicians, or other contributors who assisted in laboratory procedures and data collection and is not intended to represent an inventorship role by said laboratory personnel, technicians, or other contributors in any subject matter disclosed herein.

The term “pharmaceutically acceptable” refers to constructs, compounds and compositions which are suitable for administration to humans and/or animals without undue adverse side effects such as toxicity, irritation and/or allergic response commensurate with a reasonable benefit/risk ratio. The constructs or compounds of the present disclosure may be combined with one or more pharmaceutically-acceptable excipients, including carriers, vehicles, and diluents which may improve solubility, deliverability, dispersion, stability, and/or conformational integrity of the constructs or compounds thereof.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “individual,” “subject,” “host,” and “patient,” are used interchangeably herein and refer to any warm-blooded subject, particularly mammals, for whom diagnosis, treatment, or therapy is desired, including, but not limited to, dogs, cats, rabbits, rats, mice, guinea pigs, chinchillas, hamsters, ferrets, horses, pigs, goats, cattle, sheep, zoo animals, camels, llamas, non-human primates, including Old and New World monkeys (e.g., cynomolgus macaques, rhesus monkeys, baboons, chimpanzees, gorillas, bonobos, and orangutans), and humans.

The term “active agent” where used herein is intended to refer to a substance which possesses a biological activity relevant to the present disclosure and particularly refers to therapeutic and diagnostic substances which may be used in methods described in the present disclosure. By “biologically active” is meant the ability to modify the physiological system of a cell, tissue, or organism without reference to how the active agent has its physiological effects. Where used herein, unless otherwise noted, the term “active agent” includes pharmaceutically-acceptable salts, hydrates, solvates, and amorphous solids thereof. The recombinant gene construct(s) of the present disclosure may also be referred to herein by the term active agent(s).

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The terms “ameliorate” or “mitigate” mean that a detectable or measurable improvement occurs in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

The term “vector” as used herein refers to a macromolecule or association of macromolecules that comprises or associates with a polynucleotide and which can be used to mediate delivery of the polynucleotide to a cell. Illustrative vectors include, for example, plasmids, viral vectors (virus or the viral genome thereof), liposomes, and other gene delivery vehicles.

The term “virus” as used herein refers to a viral particle comprising a viral capsid (protein shell of a virus) and a viral genome. For example, an adeno-associated virus (AAV) refers to a viral particle comprising at least one AAV capsid protein or variant thereof and an encapsidated AAV vector genome or variant thereof.

Viral capsids typically comprise several oligomeric structural subunits made of protein called protomers. The term “encapsidate” refers to the enclosure of the genetic material, or “genome,” of the virus by the capsid. In some viruses, the capsid is enveloped, meaning that the capsid is coated with a lipid membrane known as a viral envelope.

The term “virus genome” as used herein refers to a polynucleotide sequence comprising at least one, and generally two, viral terminal repeats (e.g. inverted terminal repeats (ITRs), long terminal repeats (LTR)) at its ends. “Viral genome” may be used interchangeably herein with the terms “viral vector DNA” and “viral DNA.”

The term “recombinant viral genome” as used herein refers to a viral genome comprising a heterologous nucleic acid sequence and at least one, and generally two, viral terminal repeats at its ends. The term “recombinant virus” as used herein refers to a viral particle comprising a recombinant viral genome.

The term “heterologous” as used herein means “derived from a genotypically distinct entity” from that of the rest of the entity to which it is being compared. For example, a polynucleotide introduced by a genetic engineering technique into a plasmid or vector derived from a different species, e.g. a viral genome, is a heterologous polynucleotide. As another example, a promoter removed from its native coding sequence and operatively linked to a coding sequence to which it is not naturally linked is a heterologous promoter. As a third example, a heterologous gene product, e.g. RNA, protein, is a gene product not normally encoded by a cell in which it is being expressed.

The term “replication defective” as used herein relative to the viruses of the disclosure refers to a virus that cannot independently replicate and package its genome. For example, when a cell of a subject is infected with recombinant virions, the heterologous gene is expressed in the infected cells. However, due to the fact that the infected cells lack AAV rep and cap genes and accessory function genes, the recombinant virus is not able to replicate further. As noted above, the term “AAV” is an abbreviation for adeno-associated virus. When used herein, the term AAV may be used to refer to the virus itself or derivatives thereof, e.g. the viral capsid, the viral genome, and the like. The term “AAV” encompasses all subtypes, both naturally occurring and recombinant forms, and variants thereof except where required otherwise.

The terms “naturally-occurring” and “wild-type” refer to substances that occur in nature, or synthesized substances that are identical to natural substances. A wild-type AAV refers to an AAV or derivative thereof comprising a viral capsid that consists of viral capsid proteins that occur in nature. Non-limiting examples of naturally-occurring AAVs include AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV9, AAV10, AAV11, AAV12, rh10, avian AAV, bovine AAV, canine AAV, equine AAV, primate AAV, non-primate AAV, and ovine AAV, wherein “primate AAV” refers to AAVs that infect primates, “non-primate AAV” refers to AAVs that infect non-primate mammals, “bovine AAV” refers to AAV that infect bovids, “primate AAV” refers to AAVs that infect primates, “avian AAV” refers to AAVs that infect birds, “canine AAV” refers to AA Vs that infect canids, “ovine AAV” refers to AAVs that infect ovids, and “equine AAV” refers to AA Vs that infect equids.

The terms “AAV variant” or “variant AAV” as used herein refer to an AAV viral particle comprising a variant, or mutant, AAV capsid protein. Examples of variant AAV capsid proteins include AAV capsid proteins comprising at least one amino acid difference (e.g., amino acid substitution, insertion, or deletion) relative to a corresponding parental AAV capsid protein, i.e. an AAV capsid protein from which it was derived, a wild-type AAV capsid protein, etc., where the variant AAV capsid protein does not consist of an amino acid sequence present in a naturally occurring AAV capsid protein. In addition to differing structurally, i.e. at the sequence level, from the corresponding parental AAV, the AAV variant may differ functionally from the corresponding parental AAV.

The term “recombinant AAV” or “rAAV” as used herein refers to an AAV that comprises a heterologous polynucleotide sequence in its viral genome. In general, the heterologous polynucleotide is flanked by at least one, and generally by two naturally occurring or variant AAV inverted terminal repeat sequences (ITRs). The term rAAV vector encompasses both rAAV vector particles and rAAV vector plasmids. Thus, for example, an rAAV that comprises a heterologous polynucleotide sequence could be an rAAV that includes a nucleic acid sequence not normally included in a wild-type AAV, for example, a trans-gene (e.g. a non-AAV RNA-coding polynucleotide sequence, non-AAV protein-coding polynucleotide sequence), a non-AAV promoter sequence, or a non-AAV poly-adenylation sequence. Examples of vector constructs which may be used to deliver the coding sequences of the present disclosure include, but are not limited to, those shown in US Published Patent Applications 2021/0388030 and 2022/0183613, which are hereby expressly incorporated herein in their entireties.

As used herein, the term “expression vector” refers to a vector comprising a genetic sequence which encodes a gene product (e.g., a protein) of interest, and is used for effecting the expression of the gene product in an intended target cell. An expression vector also comprises control elements operatively linked to the encoding region to facilitate expression of the protein in the target cell. The combination of control elements and a gene or genes to which they are operably linked for expression is sometimes referred to as an “expression cassette,” a large number of which are known and available in the art or can be readily constructed from components that are available in the art.

As used herein, the term “expression” refers to the transcription and/or translation of a coding sequence, e.g. an endogenous gene, a heterologous gene, in a cell.

As used herein, the terms “gene” or “coding sequence” refer to a polynucleotide sequence that encodes a gene product such as a protein and encompasses both naturally occurring polynucleotide sequences and cDNA. A gene may or may not include regions preceding and following the coding region, e.g. 5′ untranslated (5′ UTR) or “leader” sequences and 3′ UTR or “trailer” sequences, or intervening sequences (intrans) between individual coding segments (exons).

As used herein, the term “gene product” refers the desired expression product of a polynucleotide sequence such as a polypeptide, peptide, protein or RNA including, for example, a ribozyme, short interfering RNA (siRNA), microRNA (miRNA), or small hairpin RNA (shRNA). The terms “polypeptide,” “peptide,” and “protein” are used interchangeably herein to refer to polymers of amino acids of any length. The terms also encompass an amino acid polymer that has been modified by, for example, disulfide bond formation, glycosylation, lipidation, phosphorylation, or conjugation with a labeling component.

As used herein, the terms “operatively linked” or “operably linked” refer to a juxtaposition of genetic elements on a single polynucleotide, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained. The combination of control elements and a gene or genes to which they are operably linked for expression is sometimes referred to as an “expression cassette,” a large number of which are known and available in the art or can be readily constructed from components that are available in the art. The terms “operatively linked” or “operably linked” may also be used in reference to combinations of polypeptides, for example, as subunits of a protein, or portions of a chimeric fusion protein.

The term “promoter” as used herein refers to a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species-specific may be “constitutive,” meaning continually active, or “inducible,” meaning the promoter can be activated or deactivated by the presence or absence of biotic or abiotic factors.

Any suitable promoter region can be used in the gene therapy vectors, so long as it specifically promotes expression of the gene in retinal cone cells. As used herein, “specifically” means that the promoter predominately promotes expression of the gene in retinal cone cells compared to other cell types, such that at least 70%, 75%, 80%, 85%, 90%, 91%, 92%, 93%, 94%, 95%, 96%, 97%, 97%, 98%, 99%, 99.5%, or more of expression of the gene after delivery of the vector to the eye will be in cone cells.

In certain embodiments, the gene delivery vector further comprises an enhancer element upstream of the promoter, wherein the gene is operatively linked to the enhancer element. The term “enhancer” as used herein refers to a cis-acting regulatory element that stimulates, i.e. promotes or enhances, transcription of an adjacent gene. Any suitable enhancer element can be used in the gene therapy vectors, so long as it enhances expression of the gene when used in combination with the promoter. The term “silencer” as used herein refers to a cis-acting regulatory element that inhibits, i.e. reduces or suppresses, transcription of an adjacent gene, e.g. by actively interfering with general transcription factor assembly or by inhibiting other regulatory elements, e.g. enhancers, associated with the gene. Enhancers can function (i.e., can be associated with a coding sequence) in either orientation, over distances of up to several kilobase pairs (kb) from the coding sequence and from a position downstream of a transcribed region. Enhancer sequences influence promoter-dependent gene expression and may be located in the 5′ or 3′ regions of the native gene. Enhancer sequences may or may not be contiguous with the promoter sequence. Likewise, enhancer sequences may or may not be immediately adjacent to the gene sequence. For example, an enhancer sequence may be several thousand base pairs from the promoter and/or gene sequence.

The term “termination signal sequence” as used herein refers to a genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “poly A site,” i.e. a site on an RNA transcript to which adenine residues will be added by post-transcriptional polyadenylation.

For example, the “biological activity” of an interfering RNA molecule refers to the ability of the molecule to inhibit the production of a polypeptide from a target polynucleotide sequence. The biological activity of a gene vector construct is to introduce a gene into a target cell such that the gene can be operative in the target cell.

The terms “administering” or “introducing,” as used herein in reference to gene therapy, refer to contacting a cell, tissue, or subject with a vector for the purposes of delivering a polynucleotide to the cell or to cells and or organs of the subject. Such administering or introducing may take place in vivo, in vitro or ex vivo. A vector for expression of a gene product may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

The terms “transformation” or “transfection” as used herein refer to the delivery of a heterologous DNA to the interior of a cell, e.g. a mammalian cell, an insect cell, a bacterial cell, etc. by a vector. A vector used to “transform” a cell may be a plasmid, minicircle DNA, or other vehicle. Typically, a cell is referred to as “transduced,” “infected,” “transfected,” or “transformed” dependent on the means used for administration, introduction or insertion of heterologous DNA (i.e., the vector) into the cell. The terms “transfected” and “transformed” are used interchangeably herein to refer to the introduction of heterologous DNA by non-viral methods, e.g. electroporation, calcium chloride transfection, lipofection, etc., for example as when preparing the disclosed viral vectors for use in the disclosed methods. The terms “transduced” and “infected” are used interchangeably herein to refer to introduction of the heterologous DNA to the cell in the context of a viral particle.

The term “host cell” as used herein refers to a cell which has been transduced, infected, transfected or transformed with a vector. The vector may be a plasmid, a viral particle, or a phage, for example. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments. The term “Fc region” herein is used to define a C-terminal region of an immunoglobulin heavy chain that contains at least a portion of the constant region. The term includes native sequence Fc regions and variant Fc regions.

As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided nanoparticles further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within the nanoparticle. In some embodiments, small molecules are non-polymeric. In some embodiments, in accordance with the present disclosure, small molecules are not proteins, polypeptides, oligopeptides, peptides, polynucleotides, oligonucleotides, polysaccharides, glycoproteins, proteoglycans, etc. In some embodiments, a small molecule is a therapeutic drug. In some embodiments, a small molecule is an adjuvant. In some embodiments, a small molecule is a drug.

In some embodiments, provided agents and/or compositions comprising such agents may be provided in particles. Particles as used in this context means nanoparticles or microparticles (or in some instances larger particles) which can consist in whole or in part of provided agent(s) and/or other therapeutic agent(s) as described herein. Such particles may contain the agent(s) and/or compositions in a core surrounded by a coating, including, but not limited to, an enteric coating. The agent(s) and/or compositions also may be dispersed throughout the particles. The agent(s) and/or compositions also maybe adsorbed into the particles. The particles maybe of any order release kinetics, including zero-order release, first-order release, second-order release, delayed release, sustained release, immediate release, and any combination thereof, etc. The particle may include, in addition to the agent(s) and/or compositions, any of those materials routinely used in the art of pharmacy and medicine, including, but not limited to, erodible, nonerodible, biodegradable, or nonbiodegradable material or combinations thereof. The particles maybe microcapsules which comprise one or more provided agents in a solution or in a semi-solid state. The particles may be of virtually any shape.

The term “nanoparticle,” as used herein, generally refers to a particle having a diameter, such as an average diameter, from about 1 nm, to about 5 nm, to about 10 nm, to about 50 nm up to about 1000 nm, including, for example, particles having an average diameter of 5 nm, to 10 nm, up to about 100 nm, up to about 200 nm, up to about 300 nm, up to about 400 nm, up to about 500 nm, up to about 600 nm, up to about 700 nm, up to about 800 nm, or up to about 900 nm or more. The particles can have any shape. Nanoparticles having a spherical shape are generally referred to as “nanospheres.”

The term “microparticle,” as used herein, generally refers to a particle having a diameter, such as an average diameter, from about 1 micron (micrometer) to about 100 microns, for example including particles having an average diameter from about 1 micron to about 50 microns, from about 1 micron to about 40 microns, from about 1 micron to about 30 microns, from about 1 micron to about 25 microns, from about 1 micron to about 20 microns, from about 1 micron to about 10 microns, or from about 1 to about 5 microns. The microparticles can have any shape. Microparticles having a spherical shape are generally referred to as “microspheres.”

The term M/NP (M/NPs—plural) is intended to refer to a particle of the present disclosure which may be either a microparticle or a nanoparticle, depending on the particular composition and method of particle formulation. The term M/NPs may refer to a composition comprising both nanoparticles and microparticles. The term NP refers specifically to a nanoparticle and the term MP refers specifically to a microparticle.

The term “implant,” as generally used herein, refers to a polymeric device or element that is structured, sized, or otherwise configured to be implanted, for example by injection or surgical implantation, in a specific region of the body so as to provide therapeutic benefit by releasing one or more active agents over an extended period of time at the site of implantation. For example, intraocular implants may be polymeric devices or elements that are structured, sized, or otherwise configured to be placed in the eye, for example by injection or surgical implantation, and to treat one or more diseases or disorders of the eye by releasing one or more drugs over an extended period. Intraocular implants are generally biocompatible with physiological conditions of an eye and do not cause adverse side effects. Generally, intraocular implants may be placed in an eye without disrupting vision of the eye. The M/NPs of the present disclosure may be contained within implants.

According to various embodiments, both non-biodegradable and biodegradable polymeric materials can be used in the manufacture of particles for delivering provided agent(s) and/or compositions. Such polymers maybe natural or synthetic polymers. In many embodiments, a polymer is selected based on the period of time over which release is desired.

“Biocompatible” and “biologically compatible,” as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer,” as used herein, generally refers to a polymer that will degrade or erode by enzymatic action and/or hydrolysis under physiologic conditions to smaller units or chemical species that are capable of being metabolized, eliminated, or excreted by the subject. The degradation time is a function of polymer composition, morphology, such as porosity, particle dimensions, and environment.

Examples of biodegradable polymers which can be used to form M/NPs of the present disclosure include, but are not limited to, poly(lactide-co-glycolide) (PLGA), as noted above, poly(lactic acid) (PLA), poly(L-lactic acid) (PLLA), poly(glycolic acid) (PGA), poly caprolactone (PCL), poly(lactide-co-caprolactone), poly(glycolide-co-caprolactone), poly(lactide-co-trimethylene carbonate), poly(glycolide-co-trimethylene carbonate), poly(lactide-co-glycolide-co-caprolactone), poly(lactide-co-glycolide-co-trimethylene carbonate), poly-3-hydroxybutyrate, polyanhydrides such as poly(sebacic anhydride) (PSA), and copolymers and mixtures of the above.

Bioadhesive polymers of particular interest include bioerodible hydrogels which may comprise, for example, polyhyaluronic acids, casein, gelatin, glutin, polyanhydrides, polyacrylic acid, alginate, chitosan, poly(methylmethacrylates), poly(ethylmethacrylates), poly(butylmethacrylate), poly(isobutylmethacrylate), poly(hexylmethacrylate), poly(isodecylmethacrylate), poly(laurylmethacrylate), poly(phenylmethacrylate), poly(methylacrylate), poly(isopropylacrylate), poly(isobutylacrylate), and poly(octadecylacrylate). In some embodiments, provided agents and/or compositions comprising such agents maybe contained in controlled release systems.

Other examples of biodegradable polymers which can be used to form the M/NPs of the present disclosure include, but are not limited to, poly(alkylene glycols), such as polyethylene glycol (PEG). In particular embodiments, the one or more polymers are linear PEG chains. The polymers include, for example, polyethylene glycol block polymers of the polymers listed above, such as poly(lactide-co-glycolide)-block-poly(ethylene glycol) (PLGA-b-PEG), poly(lactide)-block-poly(ethylene glycol) (PLA-b-PEG), and poly(glycolide)-block-poly(ethylene glycol) (PGA-b-PEG). Where used herein, the term PLGA also refers to poly(lactide-co-glycolide).

Representative synthetic polymers which may be used in the M/NPs of the present disclosure thus include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terephthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, and cellulose sulphate sodium salt (jointly referred to herein as “celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of natural polymers which may be used include proteins such as albumin and prolamines, for example, zein, and polysaccharides such as alginate, cellulose and polyhydroxyalkanoates, for example, polyhydroxybutyrate. Examples of non-biodegradable polymers include ethylene vinyl acetate, poly(meth)acrylic acid, polyamides, copolymers and mixtures thereof.

Pharmaceutical formulations of the present disclosure may contain the M/NPs in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are generally selected from materials which are generally recognized as safe (GRAS), and may be administered to an individual without causing undesirable biological side effects or unwanted interactions.

In some cases, the pharmaceutical formulation of the inner portion of the M/NP contains only one type of polymeric matrix for the controlled release of active agent. In other embodiments, the pharmaceutical formulation of the inner portion contains two or more different types of polymers. Pharmaceutical formulations for ocular administration may be in the form of a sterile aqueous solution or suspension of M/NPs. Acceptable solvents include, for example, water, Ringer's solution, phosphate buffered saline (PBS), and isotonic sodium chloride solution. The formulation may also be a sterile solution, suspension, or emulsion in a nontoxic, parenterally-acceptable diluent or solvent such as 1,3-butanediol. In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration. Solutions, suspensions, or emulsions for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers. Solutions, suspensions, or emulsions for ocular administration may also contain one or more tonicity agents to adjust the isotonic range of the formulation. Suitable tonicity agents are well known in the art and some examples include glycerin, mannitol, sorbitol, sodium chloride, and other electrolytes. Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as PURITE®, Allergan, Inc., Irvine, CA), phenylmercuric acetate, chlorobutanol, sorbic acid, chlorhexidine, benzyl alcohol, parabens, thimerosal, and mixtures thereof. Solutions, suspensions, or emulsions for ocular administration may also contain one or more excipients known art, such as dispersing agents, wetting agents, and suspending agents.

The term “controlled release” in this context is intended to refer to any drug-containing formulation in which the manner and profile of drug release from the formulation are controlled. This refers to immediate as well as non-immediate release formulations, with non-immediate release formulations including but not limited to sustained release and delayed release formulations.

The term “sustained release” (also referred to as “extended release”) is used in this context in its conventional sense to refer to a drug formulation that provides for gradual release of a drug over an extended period of time, and that in certain particular (but non-limiting) embodiments, although not necessarily, results in substantially constant blood levels of a drug over an extended time period. The term “delayed release” is used in this context its conventional sense to refer to a drug formulation in which there is a time delay between administration of the formulation and the release of the drug there from. “Delayed release” may or may not involve gradual release of drug over an extended period of time, and thus may or may not be “sustained release.” In some embodiments, use of a long-term sustained release implant maybe particularly suitable for treatment of chronic conditions with one or more provided agents. “Long-term” release, as used in this context, means that an implant is constructed and arranged to deliver therapeutic levels of the active ingredient for at least 7 days, and in certain non-limiting embodiments, 30-60 days. Long-term sustained release implants are well-known to those of ordinary skill in the art and include some of the release systems described elsewhere herein.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

The term “mutant” or “variant” is intended to refer to a protein, peptide, nucleic acid or organism which has at least one amino acid or nucleotide which is different from the wild type version of the protein, peptide, nucleic acid, or organism and includes, but is not limited to, point substitutions, multiple contiguous or non-contiguous substitutions, chimeras, or fusion proteins, and the nucleic acids which encode them.

An “immunoconjugate” or “antibody-drug conjugate” (ADC) is a conjugate of an antibody or antibody-derived compound with an atom, molecule, or a higher-ordered structure (e.g., with a liposome), a therapeutic agent, or a diagnostic agent.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “epitope” refers to a site on a receptor to which a construct binds. An epitope can be formed from contiguous amino acids or noncontiguous amino acids juxtaposed by tertiary folding of one or more proteins. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Also within the scope of the present disclosure are constructs in which specific amino acids have been substituted, deleted, or added. These alternations do not have a substantial negative effect on the construct's biological properties, such as (but not limited to) binding activity. For example, the construct may have amino acid substitutions in the pore-forming domain or in the receptor binding domain, such as to improve binding to the targeted receptor. Guidance concerning how to make phenotypically silent amino acid substitutions is provided in the following: Bowie et al. (Science, 247:1306-1310 (1990)); Cunningham et al. (Science, 244:1081-1085 (1989)); Ausubel (ed.) (Current Protocols in Molecular Biology, John Wiley and Sons, Inc. (1994)); Maniatis et al. (Molecular Cloning: A Laboratory Manual, Cold Spring Harbor laboratory, Cold Spring Harbor, N.Y. (1989)); Pearson (Methods Mol. Biol. 243:307-31 (1994)); and Gonnet et al. (Science, 256:1443-45 (1992)).

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser(S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent—(i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Percentage sequence identities can be determined with antibody sequences maximally aligned by the Kabat numbering convention described above. After alignment, if a particular antibody region (e.g., the entire mature variable region of a heavy or light chain) is being compared with the same region of a reference antibody, the percentage sequence identity between the disclosed and reference antibody regions is the number of positions occupied by the same amino acid in both the disclosed and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises an antibody may contain the antibody alone or in combination with other ingredients. The phrase “pharmaceutically acceptable salt” refers to pharmaceutically acceptable organic or inorganic salts of a presently-disclosed construct, or agent administered with presently-disclosed construct. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

A “human antibody” is one which possesses an amino acid sequence which corresponds to that of an antibody produced by a human or a human cell or derived from a non-human source that utilizes human antibody repertoires or other human antibody-encoding sequences. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

Antibody fragments which can be used in the present constructs as heterologous receptor binding domains to recognize specific receptors can be generated by known techniques. The antibody fragments are antigen binding portions of an antibody, such as (but not limited to) F(ab)₂, (Fab′)₂, Fab′, Fab, Fv, scFv, and other fragments described herein or otherwise contemplated in the art. Other antibody fragments include, but are not limited to: the F(ab′)₂ fragments which can be produced by pepsin digestion of the antibody molecule and the Fab′ fragments, which can be generated by reducing disulfide bridges of the F(ab′)₂ fragments. Alternatively, Fab′ expression libraries can be constructed to allow rapid and easy identification of monoclonal Fab′ fragments with the desired specificity. In certain non-limiting embodiments, the antibody fragment may be a fragment that is not an scFv fragment.

An scFv molecule comprises a V_{L} domain and a V_{H} domain. The V_{L} and V_{H} domains associate to form a target binding site. These two domains are further covalently linked by a peptide linker (L). Methods for making scFv molecules and designing suitable peptide linkers are known in the art and include (but are not limited to) those disclosed in U.S. Pat. Nos. 4,704,692 and 4,946,778, for example. An antibody fragment can be prepared by known methods, for example (but not by way of limitation), those disclosed by U.S. Pat. Nos. 4,036,945 and 4,331,647 or others as noted above.

In certain non-limiting embodiments, certain amino acid sequences of the constructs, such as (but not limited to) Fc portions of antibodies, may be varied to optimize the physiological characteristics of the sequences, such as (but not limited to) the half-life in serum. Methods of substituting amino acid sequences in proteins are widely known in the art, such as (but not limited to) by site-directed mutagenesis (e.g., Green and Sambrook, Molecular Cloning: A laboratory manual, 4^{th} ed., 2014). In certain non-limiting embodiments, the variation may involve the addition or removal of one or more glycosylation sites in the Fc sequence (see, for example but not by way of limitation, U.S. Pat. No. 6,254,868).

Chimeric amino acid sequences can be produced by recombinant DNA techniques, e.g., see Morrison et al. (Proc Natl Acad Sci, 81:6851-6855 (1984)). For example, a gene encoding a murine (or other species) monoclonal antibody molecule is digested with restriction enzymes to remove the region encoding the murine Fc, and the equivalent portion of a gene encoding a human Fc constant region is substituted. Chimeric antibodies can also be created by recombinant DNA techniques where DNA encoding murine variable regions can be ligated to DNA encoding the human constant regions, e.g., see International Patent Publication Nos. WO 87/002671 and WO 86/01533, and U.S. Pat. No. 4,816,567.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D})). Affinity can be measured by common methods known in the art, including those described herein.

The presently disclosed constructs may have a specific binding K_{D} to a targeted cell receptor of less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. Specific binding is detectably higher in magnitude and distinguishable from non-specific binding occurring to at least one unrelated target.

The presently disclosed constructs can be formulated into compositions for delivery to a mammalian subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, pH buffering agents, or adjuvants. The compositions of the present disclosure can also include ancillary substances, such as (but not limited to) pharmacological agents, cytokines, or other biological response modifiers.

Furthermore, the compositions comprising the construct(s) can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

The therapeutic composition of the present disclosure may be administered to the eye of the subject by any convenient and therapeutically-effective method, e.g. intraocularly, intravenously, or intraperitoneally. For example, the intraocular administration is intraocular may be via intravitreal injection or subretinal injection. Any suitable means for delivery of the gene therapy vector to the eye can be used, including but not limited to administering in a contact lens fluid, contact lens cleaning and rinsing solutions, eye drops, surgical irrigation solutions, ophthalmological devices, injection, iontophoresis, topical instillation on the eye, and topical instillation. The topical instillation can be administered, for example, in the form of a liquid solution, a paste, of a hydrogel. The topical instillation can be embedded, for example, in a foam matrix or supported in a reservoir. The injection into the subject's eye can be, for example, an intracameral injection, an intracorneal injection, a subconjunctival injection, a subtenon injection, a subretinal injection, an intravitreal injection, and an injection into the anterior chamber.

For intravitreal administration, the vector can be delivered in the form of a suspension. Initially, topical anesthetic is applied to the surface of the eye followed by a topical antiseptic solution. The eye is held open, with or without instrumentation, and the vector is injected through the sclera with a short, narrow, for example a 30-gauge needle, into the vitreous cavity of the eye of a subject under direct observation.

For subretinal administration, the vector can be delivered in the form of a suspension injected subretinally under direct observation using an operating microscope. Typically, a volume in a range of 1 to 200 μL, e.g. 25 μL, 50 μL, 75 μL, 100 μL, 125 μL, 150 μL, 175 μL, or 200 μL is injected subretinally. This procedure may involve vitrectomy followed by injection of vector suspension using a fine cannula through one or more small retinotomies into the subretinal space. Briefly, an infusion cannula can be sutured in place to maintain a normal globe volume by infusion (of e.g. saline) throughout the operation. A vitrectomy is performed using a cannula of appropriate bore size (for example 20- to 27-gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of vector, and (3) its controlled removal reduces the possibility of retinal tears and unplanned retinal detachment.

In practicing the disclosed methods, the vector is delivered to the eye in an amount effective to deliver the transgene to 5% or more of the subject's cone photoreceptors, for example, or to 10% or more, or to 20% or more, or to 30% or more, or to 40% or more, or to 50% or more, or to 60% or more, or to 70% or more, or to 80% or more, or to 90% or more, or to 95% or more, or to 98% or more, or 100% of the subject's cone photoreceptors to provide therapeutic benefit to the subject individual. Put another way, following the administration 5% or more of the subject's cone photoreceptors, e.g. 10% or more, 20% or more, 30% or more, 40% or more, or 50% or more, in some instance 60% or more, 70% or more, 80% or more, or 90% or more, e.g. 95%, 98%, or 100% of the cones, will comprise a sufficient amount of the polynucleotide of interest to have an impact on cone viability and/or function, e.g. to treat or prevent a disorder. In some embodiments, the transduced cones photoreceptors will be located throughout the retina. In some embodiments, the transduced cone photoreceptors will be cones in the fovea and foveola. In some embodiments, the transduced cone photoreceptors will be foveal cones, i.e. L- or M-cones located in the fovea.

Typically, an effective amount of the disclosed vector comprises an amount sufficient to produce the expression of the transgene in cells. As discussed elsewhere herein, the effective amount may be readily determined empirically, e.g. by detecting the presence or levels of transgene gene product, or by detecting an effect on the viability or function of the cone cells.

For intravitreal administration, the vector can be delivered in the form of a suspension. Initially, topical anesthetic is applied to the surface of the eye followed by a topical antiseptic solution. The eye is held open, with or without instrumentation, and the vector is injected through the sclera with a short, narrow, for example a 30-gauge needle, into the vitreous cavity of the eye of a subject under direct observation. Typically, a volume in a range of 1 to 100 μL, e.g. 25 μL, 50 μL, 75 μL, to 100 μL is injected subretinally. The composition may be delivered to the eye by intravitreal injection without removing the vitreous. Alternatively, a vitrectomy may be performed, and the entire volume of vitreous gel is replaced by an infusion of the disclosed composition.

The disclosed methods and/or compositions may be used in medicine to express a therapeutic polynucleotide in cone photoreceptors as a therapy to treat or prevent a retinal disorder. The terms “treatment,” “treating,” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues.

As discussed above, cone photoreceptors are responsible for color vision and high acuity foveal vision and are densely packed in a 1.5 mm depression located in the center of the macula of the retina, called the fovea centralis. Consistent with this, disorders associated with cone dysfunction and viability typically manifest in the macula and impact color vision and high acuity vision.

Individual doses comprising the disclosed vector are typically not less than an amount required to produce a measurable effect on the subject, and may be determined based on the pharmacokinetics and pharmacology for absorption, distribution, metabolism, and excretion (“ADME”) of the disclosed compositions or by-products thereof, and thus based on the disposition of the composition within the subject. This includes consideration of the route of administration as well as dosage amount, which can be adjusted for subretinal (applied directly to where action is desired for mainly a local effect), intravitreal (applied to the vitreous for a pan-retinal effect), or parenteral (applied by systemic routes, e.g. intravenous, intramuscular, etc.) applications. Effective amounts of dose and/or dose regimen can readily be determined empirically from preclinical assays, from safety and escalation and dose range trials, individual clinician-patient relationships, as well as in vitro and in vivo assays.

Any suitable method for producing viral particles for delivery can be used. Any concentration of viral particles suitable to effectively transduce cone cells can be administered to the eye. In one embodiment, viral particles are delivered in a concentration of at least 10¹⁰ vector genome-containing particles per mL. In various non-limiting embodiments, the viral particles are delivered in a dose having a concentration of at least 7.5×10¹⁰; or 10¹¹; or 5×10¹¹; or 10¹²; or 5×10¹²; or 10¹³; or 1.5×10¹³; or 3×10¹³; or 5×10¹³; or 7.5×9×10¹³; or 9×10¹³ vector genome-containing particles per mL. Any suitable number of administrations of the vector to the subject's eye can be made. In one embodiment, the methods comprise a single administration; in other embodiments, multiple administrations are made over time as deemed appropriate by an attending clinician.

The viral stock for delivery to the subject's eye can be treated as appropriate for delivery. The viral stock can be combined with pharmaceutically-acceptable carriers, diluents and reagents useful in preparing a formulation that is generally safe, non-toxic, and desirable, and include pharmaceutically-acceptable excipients. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide.

Compositions comprising the construct(s) can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day.

The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The dosage of an administered construct for humans will vary depending upon factors such as (but not limited to) the patient's age, weight, height, sex, general medical condition, and previous medical history. In certain non-limiting embodiments, the recipient is provided with a dosage of the construct that is in the range of from about 1 mg to about 1000 mg as a single infusion or single or multiple injections, although a lower or higher dosage also may be administered. In certain non-limiting embodiments, the dosage may be in the range of from about 25 mg to about 100 mg of the construct per square meter (m²) of body surface area for a typical adult, although a lower or higher dosage also may be administered. Non-limiting examples of dosages of the construct that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In some non-limiting embodiments, the constructs are provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9μ, about 10μ, about 15μ, about 20μ, about 25μ, about 30μ, about 35μ, about 40μ, about 45μ, about 50μ, about 60μ, about 70μ, about 75μ, about 80μ, about 90μ, about 100μ, about 125μ, about 150μ, about 175μ, about 200μ, about 250μ, about 300μ, about 350μ, about 400μ, about 500μ, about 600μ, about 700μ M, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The presently disclosed constructs can also be administered in sustained delivery or sustained release mechanisms. For example, biodegradable microspheres or capsules or other biodegradable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

In one aspect, the pharmaceutical formulations comprising constructs are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the constructs have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028.

In one aspect, the constructs are prepared with carriers that will protect the construct against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The constructs in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the constructs described herein include parenteral injection, e.g., by subcutaneous, intramuscular, or transdermal delivery. Other forms of parenteral administration include (but are not limited to) intravenous, intraarterial, intralymphatic, intrathecal, intraocular, intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The constructs can be delivered or administered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

Administration of the construct can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

Formulated compositions comprising the constructs can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents. The compositions may be administered in solution. The formulation thereof may be in a solution having a suitable pharmaceutically acceptable buffer, such as (but not limited to) phosphate, Tris(hydroxymethyl)aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of a construct of the present disclosure include a range of from about 0.001 mg/kg of the subject's body weight to about 100 mg/kg of the subject's body weight, such as but not limited to a range of from about 0.01 mg/kg to about 50 mg/kg, a range of from about 0.1 mg/kg to about 50 mg/kg, a range of from about 0.1 mg/kg to about 40 mg/kg, a range of from about 1 mg/kg to about 30 mg/kg, a range of from about 1 mg/kg to about 20 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

In some non-limiting methods, the patient is administered the construct every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The frequency of administration depends on the half-life of the construct in the circulation, the condition of the patient, and the route of administration, among other factors. The frequency can be daily, weekly, monthly, quarterly, or at irregular intervals in response to changes in the patient's condition or progression of the disorder treated. An exemplary (but non-limiting) frequency for intravenous administration is between twice a week and quarterly over a continuous course of treatment, although more or less frequent dosing is also possible. Other exemplary (but non-limiting) frequencies for intravenous administration are between once weekly or once monthly over a continuous course of treatment, although more or less frequent dosing is also possible. For subcutaneous administration, an exemplary (but non-limiting) dosing frequency is daily to monthly, although more or less frequent dosing is also possible.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

In some non-limiting embodiments, the heterologous receptor binding domain of the construct comprises a fragment of an antibody comprising an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence of the native form of the antibody fragment.

In certain non-limiting embodiments, the percent identity of two amino acid sequences (or two nucleic acid sequences) can be determined, for example, by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=(no. of identical positions÷total no. of positions)×100). The actual comparison of the two sequences can be accomplished by well-known methods, for example, using a mathematical algorithm. A specific, non-limiting example of such a mathematical algorithm is described in Karlin et al. (Proc. Natl. Acad. Sci. USA, 90:5873-5877 (1993)). Such an algorithm is incorporated into the BLASTN and BLASTX programs (version 2.2) as described in Schaffer et al. (Nucleic Acids Res., 29:2994-3005 (2001)). When utilizing BLAST and Gapped BLAST programs, the default parameters of the respective programs (e.g., BLASTN) can be used. In one non-limiting embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Several non-limiting embodiments also encompass variants of the heterologous receptor binding domains comprising one or more amino acid residue substitutions in the V_{L} domain and/or V_{H} domain thereof. Several non-limiting embodiments also encompass variants comprising one or more amino acid residue substitutions in one or more V_{L} CDRs and/or one or more V_{H} CDRs. The variants generated by introducing substitutions in the V_{H} domain, V_{H} CDRs, V_{L} domain, and/or V_{L} CDRs described herein can be tested in vitro and in vivo, for example, for its ability to bind to a corresponding receptor protein (by, e.g., immunoassays including, but not limited to, ELISAs and BIAcore).

Some non-limiting embodiments provided herein include kits. In some non-limiting embodiments, a kit can include any of the constructs as disclosed or otherwise described which is able to bind to a corresponding receptor. In some non-limiting embodiments, the construct is lyophilized. In some non-limiting embodiments, the construct is in aqueous solution, or other carrier as described herein. In some non-limiting embodiments, the kit includes a pharmaceutical carrier for administration of the construct. In some non-limiting embodiments, the kit also includes a chemotherapeutic agent. Certain non-limiting embodiments of the present disclosure include kits containing components suitable for treatments or diagnosis. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the construct may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment of certain diseases or conditions or for the diagnosis of such.

The kit or article of manufacture may comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is a construct as described herein. The label or package insert indicates that the composition is used for treating the condition of choice and further includes dosing information, for example one of the dosing regimens described herein. Moreover, the kit or article of manufacture may comprise (a) a first container with a composition contained therein, wherein the composition comprises a construct as described herein. The kit may further include other materials desirable from a commercial and user standpoint, including other buffers, diluents, filters, needles, and syringes. The term “package insert” is used to refer to instructions customarily included in commercial packages of therapeutic products, that contain information about the indications, usage, dosage, administration, combination therapy, contraindications and/or warnings concerning the use of such therapeutic products.

Certain novel embodiments of the present disclosure, having now been generally described, will be more readily understood by reference to the following examples, which are included merely for purposes of illustration of certain aspects and embodiments of the present disclosure, and are not intended to be limiting. The following detailed examples are to be construed, as noted above, only as illustrative, and not as limiting of the present disclosure in any way whatsoever. Those skilled in the art will promptly recognize appropriate variations from the various compositions, structures, components, procedures and methods.

### EXPERIMENTAL

### Materials and Methods

### Mice, Antibodies, and Other Reagents

The Cnga3^{-/-} (10), Cnga3^{-/-}/Nrl^{-/-} (14), and Nrl^{-/-} (34) mouse lines were generated as described previously. The wild-type (C57BL/6J) line was obtained from The Jackson Laboratory (Bar Harbor, ME). All mice were housed under cyclic, 12-h light-dark conditions, with ˜7-foot candles of illumination during the light cycle. Animal maintenance and experiments were approved by the local Institutional Animal Care and Use Committee (University of Oklahoma Health Sciences Center, Oklahoma City, OK) and conformed to the guidelines on the care and use of animals accepted by the Society for Neuroscience and the Association for Research in Vision and Ophthalmology (Rockville, MD). Mice of either sex were used in the experiments.

Primary antibody information is listed in Table 3. Biotinylated peanut agglutinin (PNA) was purchased from Vector Laboratories, Inc. (Burlingame, CA, USA). Horseradish peroxidase (HRP)-conjugated anti-rabbit or anti-mouse secondary antibody was obtained from Kirkegaard & Perry Laboratories Inc. (Gaithersburg, MD), fluorescent-conjugated goat anti-rabbit antibodies were purchased from Invitrogen (A21428 and A21206), Streptavidin-Cy3 was purchased from Sigma-Aldrich (S6402), and 4′,6-Diamidino-2-phenylindole (DAPI) was purchased from Sigma-Aldrich (D9542). Other reagents were obtained from Proteintech, Sigma, Cell Signaling, Invitrogen/ThermoFisher Scientific, Abcam, etc.

TABLE 3
List of primary antibodies used herein

| Antibody | Provider | Catalog No. | Dilutions | |
|---|---|---|---|---|
| M-opsin | EMD Millipore, Billerica, | AB5405 | 1:500 | (IB) |
| | MA | | 1:100 | (IF) |
| SYVN1 | Proteintech, Rosemont, IL | 13473-1-AP | 1:500 | (IB) |
| | | | 1:100 | (IF) |
| p-eIF2α | Cell signaling, Danvers, MA | 3398 | 1:500 | (IB) |
| p-IRE1α | Abcam, Cambridge, MA | ab48187 | 1:500 | (IB) |
| p-CREB | Cell signaling, Danvers, MA | 91985 | 1:500 | (IB) |
| CREB | Cell signaling, Danvers, MA | 9197 | 1:500 | (IB) |
| CREB3l3 | Novus, Centennial, CO | NBP2-16008 | 1:500 | (IB) |
| Derlin-1 | Abcam, Cambridge, MA | ab176732 | 1:500 | (IB) |
| Sel1L | LSBio, Seattle, WA | LS-C747272 | 1:500 | (IB) |
| Herpud1 | Thermo Fisher Scientific, | PA5-29469 | 1:500 | (IB) |
| | Waltham, MA | | | |
| CHOP | Cell signaling, Danvers, MA | 2985 | 1:500 | (IB) |
| p53 | Proteintech, Rosemont, IL | 10442-1-AP | 1:500 | (IB) |
| Ubiquitin | Proteintech, Rosemont, IL | 10201-2-AP | 1:500 | (IB) |
| TBP | Thermo Fisher Scientific, | MA1-10883 | 1:1000 | (IB) |
| | Waltham, MA | | | |
| β-actin | Abcam, Cambridge, MA | ab6276 | 1:2000 | (IB) |

### Construction of AAV5-IRBP/GNAT2-Syvn1 Vectors and Intraocular Injection

The Syvn1 mouse cDNA was cloned into an AAV vector plasmid containing the chimeric cone-specific IRBP/GNAT2 promotor, which has been demonstrated to direct the expression of transgene specifically in cones. The vector construct was subsequently packaged into AAV serotype-5 by plasmid transfection into HEK293T cells. The resulting AAV5-IRBP/GNAT2-Syvn1 was purified and tittered. Intraocular injections were performed under a surgical operating microscope (Zeiss, Thornwood, NY). Briefly, after anesthesia on ice for 2-3 min, one eye of a pup at postnatal 5 (P5) was opened by cutting along the fused junctional epithelium. A 30-gauge sharp disposable needle (BD Biosciences, Franklin Lakes, NJ) was used to punch a hole immediately below the limbus. Through the hole, one microliter of AAV5-IRBP/GNAT2-Syvn1 was injected intraocularly with a NanoFil microsyringe injector system with a 33-gauge blunt needle (Hamilton Co, Reno, NV). Only eyes with no apparent surgical complications were retained for further evaluation.

### Eye Preparation, Immunofluorescence Labeling, and Confocal Microscopy

Retinal whole mounts or cross sections were prepared for immunofluorescence labeling. For retinal whole mount preparations, eyes were enucleated, marked at the superior pole with a green dye, and fixed in 4% paraformaldehyde (PFA; Polysciences, Inc., Warrington, PA) for 30 min at room temperature, followed by removal of the cornea and lens. The eyes were then fixed in 4% paraformaldehyde in PBS for 4-6 h at room temperature, and retinas were isolated, and the superior portion was marked for orientation with a small cut. For retinal cross-sections, mouse eyes were enucleated (the superior portion of the cornea was marked with green dye prior to enucleation) and fixed in Prefer (Anatech Ltd., Battle Creek, MI) for 25-30 min at room temperature. Paraffin sections (5-μm thickness) passing vertically through the retina (along the vertical meridian passing through the optic nerve head) were prepared using a Leica microtome (Leica Biosystems, Buffalo Grove, IL).

Immunofluorescence labeling was performed. Briefly, retinal whole mounts or sections were blocked with Hanks' balanced salt solution containing 5% BSA and 0.5% Triton X-100 for 1 h at room temperature or overnight at 4° C. Prior to blocking, antigen retrieval was performed in 10 mM sodium citrate buffer (pH 6.0) in a 70° C. water bath. Primary-antibody incubation was performed for 2 h at room temperature or overnight at 4° C. (see Table 1 for antibody information). Slides were mounted and coverslipped after fluorescence-conjugated secondary-antibody incubation and wash steps. Immunofluorescence labeling was then imaged using an Olympus FV1000 confocal laser-scanning microscope and Fluo View imaging software (Olympus, Melville, NY). For evaluations of cone OS protein cellular localization, confocal images of 10 layers were stacked with the Z-stack function in the ImageJ software (https://imagej.nih.gov/ij/) to obtain a maximal immunofluorescence density. Fluorescence density levels of the immunolabeling in the OS, IS, outer nuclear layer, and outer plexiform layer were measured, and the density levels at each region relative to the total fluorescence density were calculated and averaged from at least three sections per eye from at least five animals per condition.

### TUNEL Assay

Terminal deoxynucleotidyl transferase dUTP nick-end labeling (TUNEL) was performed to analyze photoreceptor apoptotic death, using paraffin-embedded retinal sections and an in situ cell death fluorescein detection kit (Roche Applied Science, Ref. 11684795910). Immunofluorescence labeling was imaged using an Olympus FV1000 confocal laser-scanning microscope, and TUNEL-positive cells in the ONL passing through the optic nerve were counted and averaged from at least three sections per eye from at least four animals per condition.

### Retinal Protein Preparation, SDS-PAGE, and Western Blot Analysis

Retinal protein preparation, SDS-PAGE, and western blot analysis were performed. Briefly, retinas were homogenized in homogenization buffer A [0.32 M sucrose, 20 mM 4-(2-hydroxyethyl)-1-piperazineethanesulfonic acid (HEPES), pH 7.4, and 3 mM EDTA containing protease and phosphatase inhibitors] (Roche Applied Science, catalog no. 04693159001 and 04906837001, respectively), and homogenates were centrifuged at 1,200 g for 10 min at 4° C. The resulting supernatant was then centrifuged at 13,000 rpm for 35 min at 4° C. to separate cytosolic (supernatant) and membrane (pellet) fractions. The resulting pellet in the first step and the membrane pellet in the second step were resuspended in homogenization buffer B (0.32 M sucrose, 20 mM HEPES, pH 7.4, 3 mM EDTA, and 0.1% Triton X-100 containing protease and phosphatase inhibitors, as described above), sonicated twice for 15 s on ice at medium speed using a Masonix XL2000 ultrasonic cell disruptor with a 30-s recovery between disruptions, and incubated for 1 h at 4° C. with gentle agitation. After incubation, the homogenate was centrifuged at 13,000 rpm for 35 min at 4° C. The resulting supernatant was used as the nuclear and membrane fraction. All protein concentrations were determined by a protein-assay kit from Bio-Rad Laboratories. Retinal protein samples (20 μg protein per sample) were then subjected to SDS-PAGE and transferred to PVDF membranes, followed by blocking in 5% nonfat milk or 5% bovine serum albumin (BSA) for 1 h at room temperature. Immunoblots were incubated with primary antibody overnight at 4° C. After washing in Tris-buffered saline with 0.1% Tween 20, immunoblots were incubated with horseradish peroxidase-conjugated secondary antibody (1:20,000) for 1 h at room temperature. SuperSignal® West Dura Extended Duration chemiluminescent substrate (Thermo Fisher Scientific, catalog no. 34076) was used to detect binding of the primary antibodies to their cognate antigens. A Li-Cor Odyssey CLx Imager and Li-Cor software (Li-Cor Biosciences, Lincoln, NE, USA) were used for detection and densitometric analysis.

### Statistical Analysis

The results are expressed as means #SEM of the number of mice or the number of assays. Power analysis was performed to choose the sample size. The analysis indicates that a sample size of 3-5 mice/group for evaluations of retinal degeneration in the mouse retinas will provide at least 80% power (1-β) for a two-sided, two-sample t-test at a 0.05 alpha level. One-way ANOVA was used for significance within sets of data, followed by Dunn's multiple comparisons test. Unpaired Student's t-test/Mann-Whitney test was used for differences between two groups of data. Differences were considered statistically significant when p<0.05. Data were analyzed and graphed using GraphPad Prism® software (GraphPad Software, San Diego, CA).

### Results

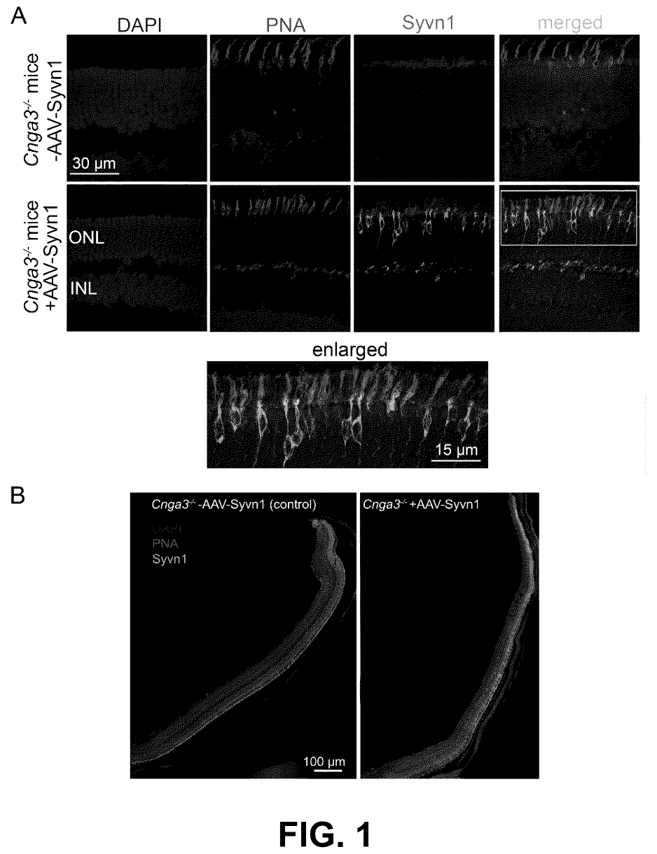

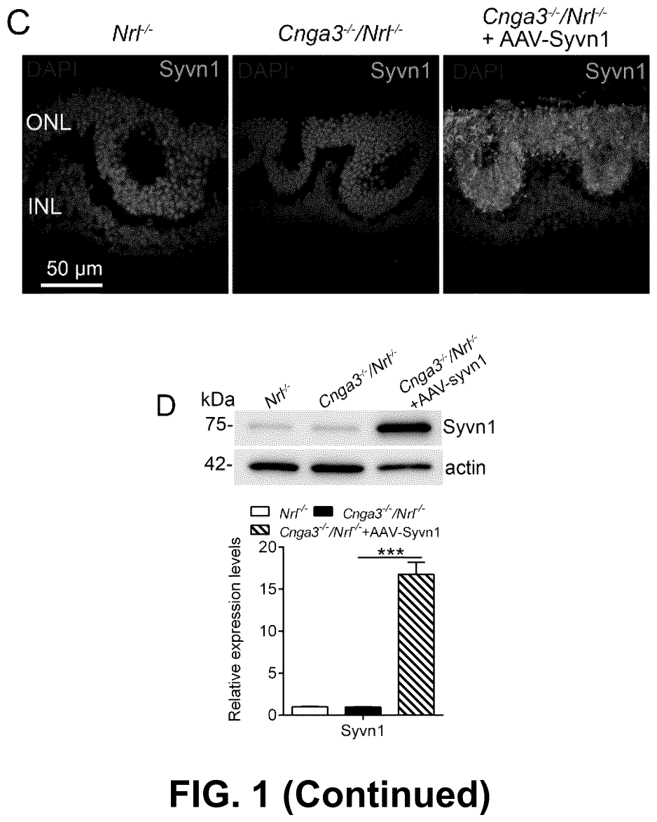

SYVN1 is overexpressed in cones of CNG channel-deficient mice after intraocular injection with AAV5-IRBP/GNAT2-Syvn1 Expression levels of SYVN1 in CNG channel-deficient cones/retina after injection of AAV5-IRBP/GNAT2-Syvn1 was evaluated in Cnga3^{-/-} and Cnga3^{-/-}/Nrl^{-/-} mice. Cnga3-mice received AAV5-IRBP/GNAT2-Syvn1 (1×10¹⁰ vector genomes, 1 μl) at P5 and were evaluated for retinal expression/localization of SYVN1 at 4 months by immunoblotting using anti-SYVN1 antibody. SYVN1 was detected in the inner segments of the retinal sections, which is consistent with its ER localization. Immunolabeling was increased in mice that received treatment with AAV-Syvn1, compared with untreated controls. Co-labeling with PNA confirmed overexpression of SYVN1 in cones (FIG. 1, Panel A). Cnga3^{-/-}/Nrl^{-/-} mice were used for the evaluation by immunoblotting. Mice lacking NRL, a rod-specific neural retina leucine zipper transcriptional factor, have a cone-dominant retina. Cnga3^{-/-}/Nrl^{-/-} mice exhibit early-onset cone degeneration and impaired cone function similar to that seen in the single knockout line (13, 14), thus allowing us to examine the cellular/biochemical alterations in CNG channel-deficient cones (because cones comprise only 3-5% of total number of photoreceptor population in a mammalian retina using immunoblotting. Cnga3^{-/-}/Nrl^{-/-} mice were intraocularly injected with AAV5-IRBP/GNAT2-Syvn1 at P5 and were evaluated at P40. Immunoblotting showed that the expression level of SYVN1 increased by about 15-fold in mice treated with AAV-Syvn1 virus, compared with untreated controls (FIG. 1, Panel B).

Cone Density is Increased in Cnga3^{-/-} Mice after Treatment with AAV5-IRBP/GNAT2-Syvn1

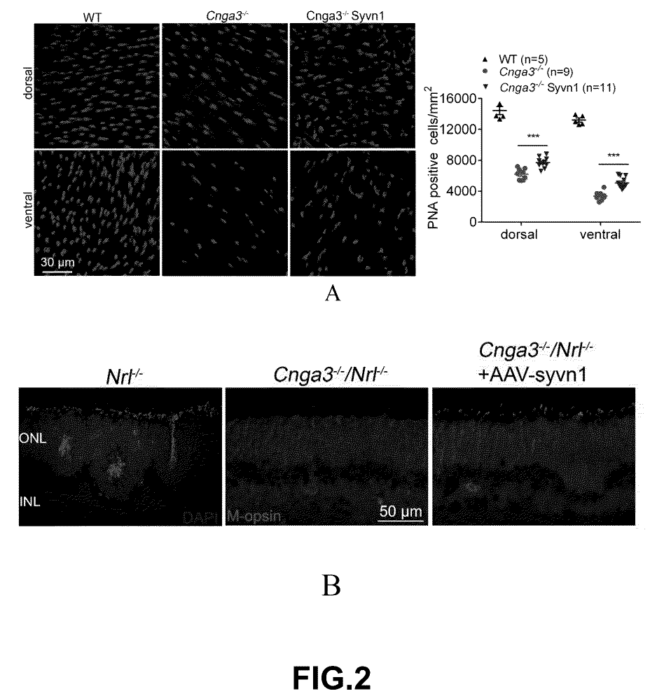

CNG channel-deficient mice show progressive cone degeneration, manifested as reduced cone density. To evaluate the effects of SYVN1 overexpression, P5 Cnga3^{-/-} mice were injected with AAV5-IRBP/GNAT2-Syvn1 and evaluated for cone density at 4 months by PNA labeling of retinal whole mounts. Treatment with AAV5-IRBP/GNAT2-Syvn1 increased cone density by 30% and 60% in the dorsal and ventral areas, respectively, compared with untreated controls (FIG. 2, Panel A). In a separate experiment, cone survival in Cnga3^{-/-}/Nrl^{-/-} mice was assessed by M-opsin labeling. P5 Cnga3^{-/-}/Nrl^{-/-} mice received AAV5-IRBP/GNAT2-Syvn1 and were evaluated at P40. M-opsin expression was nearly undetectable in retinal sections of Cnga3^{-/-}/Nrl^{-/-} mice, compared with Nrl^{-/-} controls. However, M-opsin expression was more apparent in retinas of Cnga3^{-/-}/Nrl^{-/-} mice treated with AAV-Syvn1, compared with untreated controls (FIG. 2, Panel B).

Cone Apoptosis is Reduced in Cnga3^{-/-}/Nrl^{-/-} Mice after Treatment with AAV5-IRBP/GNAT2-Syvn1

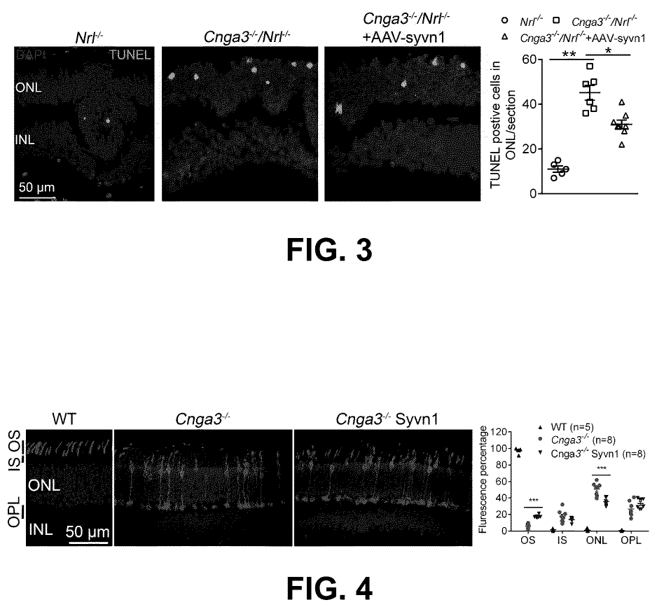

Apoptotic cone death has been well characterized in CNG channel-deficient mice. Here, the effects of SYVN1 overexpression on this process were examined. P5 Cnga3^{-/-}/Nrl^{-/-} mice received AAV5-IRBP/GNAT2-Syvn1 and were evaluated at P40. Cone death/apoptosis was evaluated by TUNEL labeling in retinal sections. The number of TUNEL-positive cells was significantly reduced in Cnga3^{-/-}/Nrl^{-/-} mice treated with AAV-Syvn1, compared with untreated controls (FIG. 3).

Outer Segment Localization of Cone Opsin is Increased in Cnga3^{-/-} Mice after Treatment with AAV5-IRBP/GNAT2-Syvn1

Mislocalization of cone outer segment (OS) proteins has been well characterized in CNG channel-deficient mice. Here, the effects of SYVN1 overexpression on this protein localization were examined. P5 Cnga3^{-/-} mice received subretinal injections of AAV5-IRBP/GNAT2-Syvn1 and were evaluated for M-opsin localization in the retina at 4 months by immunolabeling. Cnga3^{-/-} mice show drastic mislocalization of M-opsin, manifested as decreased levels of M-opsin localized to the OS and increased levels in the inner segment (IS), outer nuclear layer (ONL), and outer plexiform layer (OPL) (FIG. 4). Treatment with AAV5-IRBP/GNAT2-Syvn1 significantly improved OS localization of M-opsin in Cnga3-mice, compared with untreated controls (FIG. 4). The total OS immunofluorescence density level of M-opsin labeling was increased by about 1.5-fold and the total ONL immunofluorescence density level was decreased by about 35% in Cnga3^{-/-} mice after viral injection.

ER Stress Responses are Reduced in Retinas of Cnga3^{-/-}/Nrl^{-/-} Mice after Treatment with AAV5-IRBP/GNAT2-Syvn1

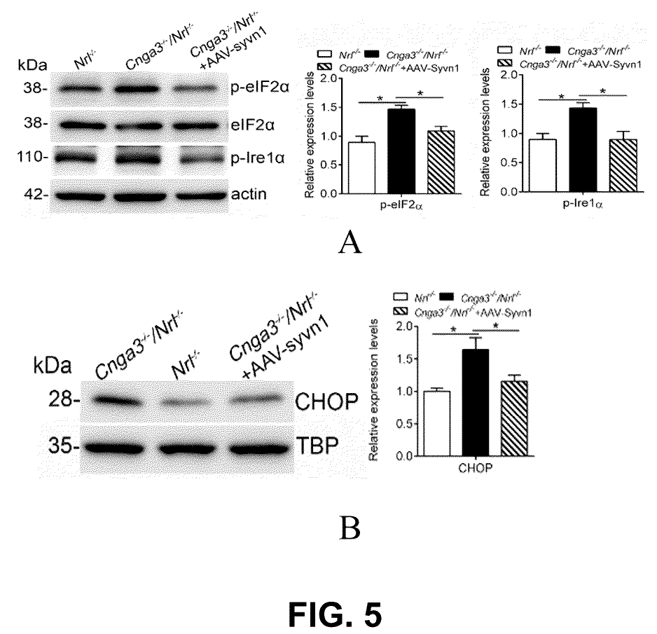

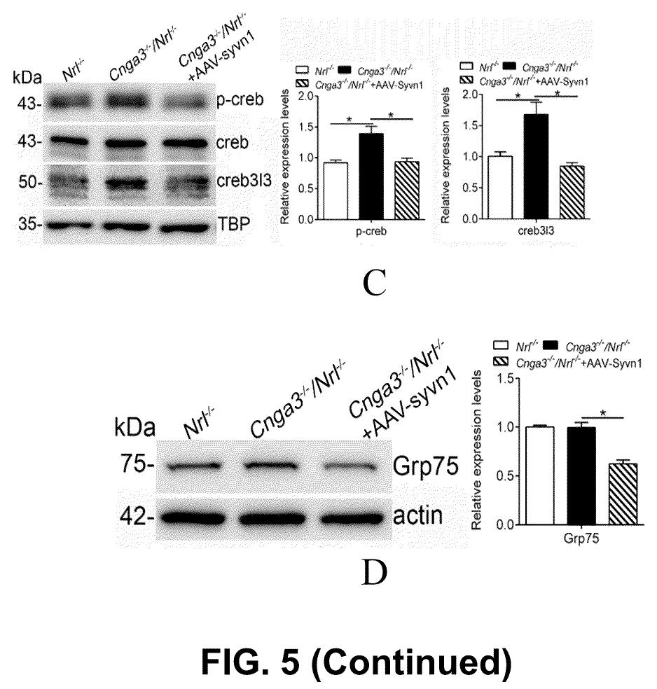

ER stress has been associated with cone degeneration in CNG-channel deficient retinas. Here, the effects of SYVN1 overexpression on ER stress and its involvement in downstream death signaling pathways were examined. P5 Cnga3^{-/-} Nrl^{-/-} mice were intraocularly injected with AAV5-IRBP/GNAT2-Syvn1 and evaluated at P40. The two ER stress markers, eIF2α (eukaryotic translation initiation factor 2 alpha) and IRE1α (serine/threonine-protein kinase/endoribonuclease), were examined to evaluate the activity of ER stress pathways. Phospho-eIF2a and phospho-IRE1α levels were significantly increased in Cnga3^{-/-}/Nrl^{-/-} mice, compared with Nrl^{-/-} controls, and treatment with AAV5-IRBP/GNAT2-Syvn1 abolished these elevations (FIG. 5, Panel A). The effects of SYVN1 overexpression were further examined by evaluating CHOP expression. CHOP is a member of the C/EBP (CCAAT enhancer-binding protein) transcription factor family induced in ER stress and is involved in ER stress-associated apoptosis. Elevated expression/activation of CHOP in CNG channel-deficient retinas has previously been shown. The immunoblotting results showed that treatment with AAV-Syvn1 completely abolished CHOP activation in Cnga3^{-/-}/Nrl^{-/-} retinas (FIG. 5, Panel B). CAMP-response element binding protein (CREB) and cAMP-responsive element-binding protein 3-like 3 (CREB313) are transcription factors activated by ER stress signaling. Increased expression of phospho-CREB and CREB313 in CNG channel-deficient retinas has been demonstrated. Immunoblotting results showed that treatment with AAV-Syvn1 completely abolished elevation of phospho-CREB and CREB313 in retinas of Cnga3^{-/-}/Nrl^{-/-} mice (FIG. 5, Panel C). GRP75 is a heat shock protein and localizes to the mitochondria and the endoplasmic reticulum. This protein plays a role in cell proliferation, stress response and maintenance of mitochondria. It has been shown to modulate ER-mitochondria coupling and accelerates Ca²⁺-dependent endothelial cell apoptosis in diabetic retinopathy. It was found that treatment with treatment with AAV-Syvn1 significantly reduced expression of GRP75, although there was no difference between untreated Cnga3^{-/-}/Nrl^{-/-} mice and Nrl^{-/-} controls (FIG. 5, Panel D).

Increased Expression of ER Retrotranslocation Proteins is Observed in Retinas of Cnga3^{-/-}/Nrl^{-/-} Mice after Treatment with AAV5-IRBP/GNAT2-Syvn1

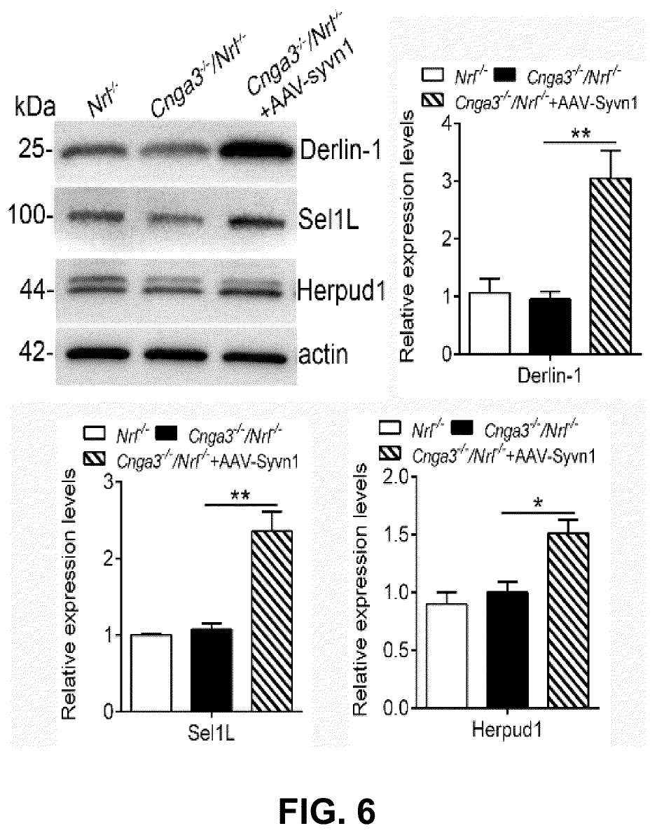

Previous studies showed elevated expression of the ER retrotranslocation proteins in Cnga3^{-/-}/Nrl^{-/-} mice, including SYVN1, SEL1L, and HERPUD1, when ER stress was reduced. Here it was examined how SYVN1 overexpression related to expression levels of these retrotranslocation proteins. P5 Cnga3^{-/-}/Nrl^{-/-} mice were injected with AAV5-IRBP/GNAT2-Syvn1 and evaluated at P40 for ER retrotranslocation protein expression levels by immunoblotting. The analysis revealed that ER retrotranslocation protein expression levels were significantly increased in Cnga3^{-/-}/Nrl^{-/-} mice after treatment with AAV-Syvn1. Expression of DERL1, SEL1L, and HERPUD1 were increased by about 2-fold, 1-fold, and 50%, respectively, in AAV-Syvn1-treated Cnga3^{-/-}/Nrl^{-/-} mice, compared with untreated controls (FIG. 6).

Enhanced Ubiquitination and Proteasome Degradation is Observed in Retinas of Cnga3^{-/-}/Nrl^{-/-} Mice after Treatment with AAV5-IRBP/GNAT2-Syvn1

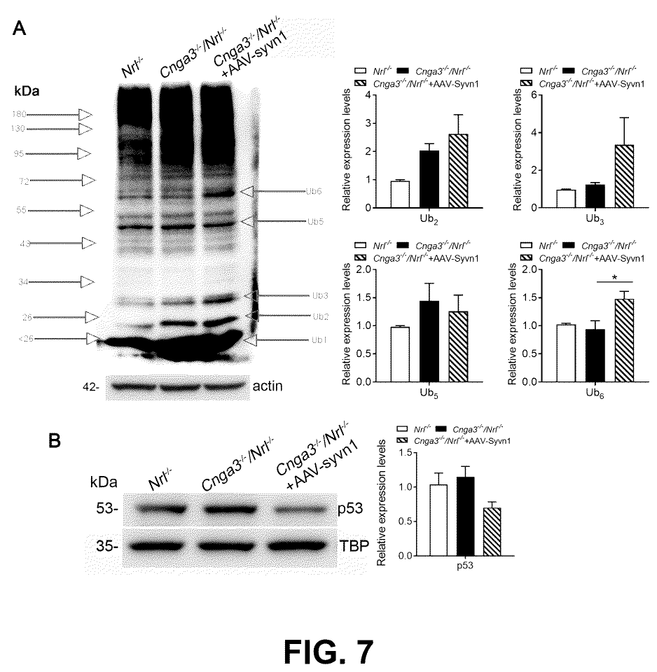

Based on the critical role of SYVN1 in protein ubiquitination/ERAD, the effects of its overexpression on protein ubiquitination/proteasome degradation in the retina were examined. P5 Cnga3^{-/-}/Nrl^{-/-} mice were intraocularly injected with AAV5-IRBP/GNAT2-Syvn1 and evaluated at P40 for protein ubiquitination by immunoblotting using anti-ubiquitin (Ub) antibody. The antibody detected free/monomeric Ub and several polyubiquitin chains, including Ub₂, Ub₃, Ub₅, Ub₆ and Ub₁₆. It was found that the overall protein ubiquitination (Ub) was enhanced in Cnga3^{-/-}/Nrl^{-/-} retinas, compared with that in Nrl^{-/-} controls, and that treatment with AAV-Syvn1 led to further elevation (FIG. 7, Panel A). Expression levels of Ub₂ and Ub₃ were increased in Cnga3^{-/-}/Nrl^{-/-} retinas, compared to Nrl^{-/-} control retinas, and treatment with AAV-Syvn1 led to their further elevation. Ub₆ expression was significantly reduced in Cnga3^{-/-}/Nrl^{-/-} retinas, compared to Nrl^{-/-} control retinas, while treatment with AAV-Syvn1 reversed this reduction. There was no significant change in the expression levels of Ub₅ and Ub₁₆ between Cnga3^{-/-}/Nrl^{-/-} and Nrl^{-/-} mice and after the viral treatment (FIG. 7, Panel B). p53, a tumor suppression protein, has been shown to be recruited by SYVN1 for ubiquitination and proteasomal degradation. Thus, p53 expression in AAV-Syvn1-treated Cnga3^{-/-}/Nrl^{-/-} retinas was examined to estimate the effects of SYVN1 overexpression on proteasome activity. Results show that the p53 expression was reduced by 40% in AAV-Syvn1-treated Cnga3^{-/-}/Nrl^{-/-} retinas, compared to untreated controls (FIG. 7, Panel B). In addition, the expression level of pIRE1α, which is also a known endogenous substrate of ERAD, was significantly reduced (FIG. 5, Panel A).

### Discussion

SYVN1 is Overexpressed in Cones after Intraocular Injection of AAV5-IRBP/GNAT2-Syvn1.

Overexpression of SYVN1 in CNG channel deficiency mice after subretinal injection of AAV5-IRBP/GNAT2-Syvn1 was demonstrated by both immunofluorescence labeling and immunoblotting. Immunofluorescence labeling of retinal sections prepared from Cnga3^{-/-} mice injected with AAV5-IRBP/GNAT2-Syvn1 showed SYVN1 signal in the photoreceptor inner segment, which is consistent with the feature of ER resident of this protein. Labeling with a cone marker demonstrated the increased labeling of SYVN1 signal in cones. It was also detected in the ONL and OPL areas, suggesting an outcome of overexpression. Immunoblotting using retinas prepared from the cone-dominant mice, Cnga3^{-/-}/Nrl^{-/-}, showed that SYVN1 expression in mice treated with AAV5-IRBP/GNAT2-Syvn1 was increased by about 15-fold, compared with untreated controls. These data demonstrate that injection of AAV5-IRBP/GNAT2-Syvn1 is an effective approach to overexpress SYVN1 in photoreceptors.

### Effects of SYVN1 Overexpression in CNG Channel Deficiency.

Overexpression of SYVN1 leads to effective cone protection. Cone density/survival evaluated by PNA labeling and M-opsin labeling was significantly increased after subretinal delivery of AAV5-IRBP/GNAT2-Syvn1. Cone apoptosis was significantly reduced in CNG channel-deficient mice and cone opsin localization to the outer segments was increased in CNG channel-deficient mice after treatment with AAV-Syvn1. These measurements show improved cone survival/health in CNG channel deficiency resulting from overexpression of SYVN1/improved ER retrotranslocation. Overexpression of SYVN1 leads to ER preservation/reduced ER stress, manifested as reduced levels of eIF2a and IRE1α, reduced CREB signaling, and reduced death signaling/CHOP signaling. It is reasonable to believe that the overall reduced ER stress contributed to the improved cone survival/protection and outer segment protein localization. Further evaluation shows that SYVN1 overexpression leads to increased expression levels of ER retrotranslocation proteins, including DERL1, SEL1L and HERPUD1. This is an interesting observation, suggesting a positive feedback regulation of expression among the different components of the retrotranslocon complex, though how this regulation takes place remains unclear and merits further investigation. Further evaluation also shows enhanced ubiquitination and proteasome activity, manifested as increased levels of monomeric Ub and several polyubiquitin chains and increased degradation of the ubiquitination/ERAD target proteins, including P53 and Ire1α. As a primary sensor for UPR/ER stress, IRE1α is upregulated in response to ER stress. Ire1α is also a known target of ERAD. Thus, the reduced level of Ire1α in CNG channel-deficient retinas after treatment with AAV-Syvn1 might be a result of both reduced production and increased degradation due to enhanced ERAD activity. It is convincing that enhanced ubiquitination/ERAD is a result of SYVN1 overexpression and enhanced expression of other retrotranslocon components. Nevertheless, the overall enhanced ER retrotranslocation and ubiquitination/proteasome activity after overexpression of SYVN1 very likely contributed to the reduced ER stress in CNG channel deficiency and improved localization of protein to cone outer segments.

### SYVN1 Overexpression in Neuronal/Photoreceptor Protection.

ERAD is critical for ER proteostasis control and has been recognized as an important regulation mechanism for cell protection. Promoting ERAD has reduces neuronal degeneration, including retinal degeneration. Upregulation/overexpression of SYVN1 suppresses ER stress and neuronal cell death and delays progression of Alzheimer's and Parkinson's diseases. The role of SYVN1/ERAD in preservation of photoreceptors has been studied using different animal models. Overexpression of SYVN1 suppresses retinal degeneration in drosophila and diabetic rat. Overexpression of SYVN1 also inhibits inflammatory cytokine secretion from Müller cells in a diabetic mouse model. Moreover, SYVN1 overexpression promotes ubiquitination and degradation of STAT3, prevents neovascularization, and extends physiologic retinal vascular development in the retinal tissues of a mouse model of oxygen-induced retinopathy. The present work for the first time demonstrated the role of SYVN1 overexpression in photoreceptor protection in mouse models of cone degeneration. Consistent with findings showing the role of SYVN1 in neuroprotection and photoreceptor protection, increased proteasomal activity has been shown to support photoreceptor survival in retinal degeneration, and proteasomal insufficiency might be a general mechanism across multiple degenerative diseases. Thus, efficient ERAD is critical for maintenance of ER/cellular homeostasis and cell survival.

### ER Ca²⁺ Homeostasis and Expression Levels of ER Retrotranslocon Proteins in CNG Channel Deficiency.

The present work demonstrates that SYVN1 overexpression protects cones in CNG channel deficiency. Interestingly, similar findings were obtained with approaches to preserve ER Ca²⁺ levels. CNG channel-deficient cones show impaired cellular/ER Ca²⁺ homeostasis. Preservation of ER Ca²⁺ level by deletion of ER calcium channels IP₃R1 or RyR2 reduces ER stress/cone death and improves cone protein trafficking, accompanied by increased expression of ER retrotranslocation protein. How preservation of ER Ca²⁺ levels leads to increased expression of the retrotranslocation proteins remains unclear at this time. However, these observations suggest that the mechanisms in which preservation of ER Ca²⁺ induces ER preservation/cone protection may involve, at least in part, an improved ER retrotranslocation/ERAD. Moreover, treatment with a chemical chaperone, which reduces ER stress in photoreceptors, also increases expression of these proteins. Like chaperone proteins, chemical chaperones are a class of small molecules that function to enhance the folding and/or stability of proteins through a variety of mechanisms, thereby improving protein trafficking and relieving ER stress. How administration of a chaperone leads to increased expression of the retrotranslocation proteins remains unclear at this time. However, this observation suggests that the mechanisms in which administration of a chaperone induces ER preservation/cone protection may involve, at least in part, an improved ER retrotranslocation and ERAD.

### The Role of ER Retrotranslocation/ERAD in Cone Protection in CNG Channel Deficiency.

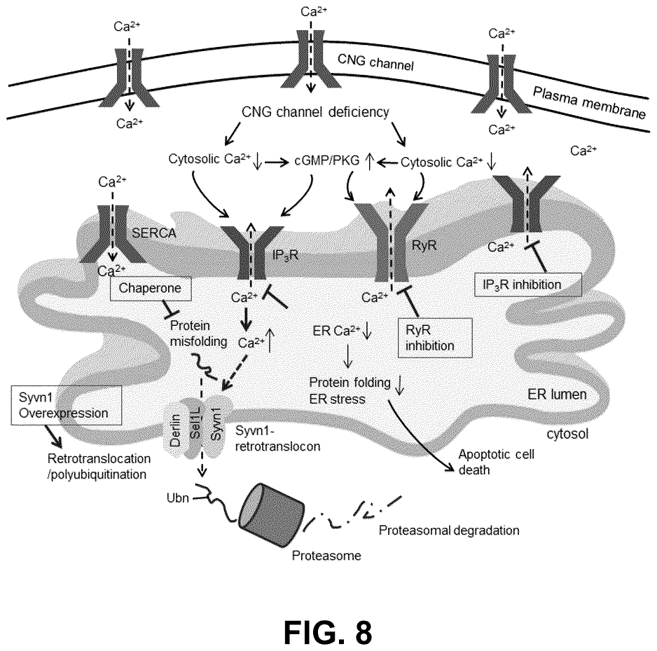

In CNG channel deficiency, reduced cytosolic Ca²⁺ levels stimulate ER Ca²⁺ channels, leading to increased Ca²⁺ release from the ER. This compensatory response potentially leads to a reduction of ER Ca²⁺ store/impaired ER Ca²⁺ homeostasis. Moreover, reduced cytosolic Ca²⁺ levels also stimulate cGMP/PKG signaling, which is a strong inducer for the ER Ca²⁺ channels, impairing ER Ca²⁺ store/homeostasis. Impaired ER Ca²⁺ store/homeostasis inhibits ER protein processing/protein folding, presumably via the regulation of the Ca²⁺-dependent ER chaperone proteins, leading to ER stress/cell death. Thus, suppressing ER Ca²⁺ channel activity improves protein trafficking in cones and reduces ER stress/cell death. The previous findings and results from the present work support that ER retrotranslocation/ERAD in cone photoreceptors is regulated at least by the following three factors: (1) ER Ca²⁺ store/homeostasis, (2) function of ER chaperones, and (3) expression levels of the retrotranslocon components. Increased Ca²⁺ stores in the ER, enhanced protein folding/processing, and enhanced expression of the ER retrotranslocon components will facilitate ER retrotranslocation/ERAD and relieve ER stress, leading to preservation of photoreceptors (FIG. 8).

In summary, the present work demonstrates that SYVN1 overexpression in cones of CNG channel-deficient mice can be achieved by subretinal injection of AAV5-IRBP/GNAT2-Syvn1. Overexpression of SYVN1 reduces ER stress/cone death, improves outer segment localization of cone opsin, and increases cone density in CNG channel-deficient mice. Overexpression of SYVN1 also leads to increased expression of ER retrotranslocon components and enhances ubiquitination/proteasome degradation in the channel-deficient retinas. The present work demonstrates the role of SYVN1/ERAD in cone preservation in CNG channel deficiency and provides insight into how restoration of ER Ca²⁺ level/homeostasis reduces ER stress/cone death. Findings from this work support the strategy of promoting ERAD for cone protection.

While the present disclosure has been described in connection with certain variants, compositions, and methods of production and application thereof so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the formulation of the various compositions described herein, the methods described herein or in the steps or the sequence of steps of the methods described herein without departing from the spirit and scope of the present disclosure.

What is claimed is:

1. A method of treating a cone photoreceptor cell-associated disorder in a subject in need of such therapy, comprising:
administering to the eye of the subject a recombinant vector comprising:
(a) a coding sequence encoding a synoviolin 1 (SYVN1) protein; and
(b) a promoter region, wherein the promoter region is specific for cone photoreceptor cells;
wherein the coding sequence is operatively linked to the promoter region; and
wherein in vivo expression of the SYVN1 protein in the cone photoreceptor cells of the subject serves to treat the cone photoreceptor cell-associated disorder in the subject.

2. The method of claim 1, wherein the recombinant vector further comprises an enhancer element upstream of the promoter, wherein the coding sequence is operatively linked to the enhancer element.

3. The method of claim 1, wherein the SYVN1 protein encoded by the coding sequence is a human SYVN1 protein or an orthologue having at least 90% identity to the human SYVN1 protein.

4. The method of claim 3, wherein the SYVN1 protein encoded by the coding sequence has at least 90% identity to SEQ ID NO:1.

5. The method of claim 3, wherein the human SYVN1 protein comprises the amino acid sequence of SEQ ID NO: 1.

6. The method of claim 1, wherein the coding sequence comprises a sequence that is at least 90% identical to SEQ ID NO:2.

7. The method of claim 1, wherein the cone photoreceptor cell-associated disorder is selected from the group consisting of rod-cone dystrophy, cone-rod dystrophy, progressive cone dystrophy, retinitis pigmentosa (RP), Stargardt's Disease, Leber hereditary optic neuropathy (LHON), Leber congenital amaurosis (LCA), Best disease, adult vitelliform macular dystrophy (AVMD), X-linked retinoschisis, blindness, a color vision disorder, color blindness, blue cone monochromacy, achromatopsia, incomplete achromatopsia, protan defects, deutan defects, tritan defects, disorders of the central macula, dry age-related macular degeneration (dry AMD), wet age-related macular degeneration (wet AMD), geographic atrophy, retinal telangiectasia, macular telangiectasia, Coats' disease, diabetic retinopathy, retinal vein occlusions, retinal ischemia, Familial Exudative Vitreoretinopathy (FEVR), glaucoma, Sorsby's fundus dystrophy, and combinations thereof.

8. A method of treating a cone photoreceptor cell-associated disorder in a subject in need of such therapy, comprising:
administering to the eye of the subject a recombinant vector comprising:
(a) a coding sequence encoding a synoviolin 1 (SYVN1) protein;
(b) a promoter region, wherein the promoter region is specific for cone photoreceptor cells; and
(c) an enhancer element upstream of the promoter;
wherein the coding sequence is operatively linked to the promoter region and to the enhancer element; and
wherein in vivo expression of the SYVN1 protein in the cone photoreceptor cells of the subject serves to treat the cone photoreceptor cell-associated disorder in the subject.

9. The method of claim 8, wherein the SYVN1 protein encoded by the coding sequence comprises a human SYVN1 protein or an orthologue having at least 90% identity to SEQ ID NO: 1.

10. The method of claim 9, wherein the human SYVN1 protein comprises the amino acid sequence of SEQ ID NO:1.

11. The method of claim 8, wherein the coding sequence comprises a sequence that is at least 90% identical to SEQ ID NO:2.

12. The method of claim 8, wherein the cone photoreceptor cell-associated disorder is selected from the group consisting of rod-cone dystrophy, cone-rod dystrophy, progressive cone dystrophy, retinitis pigmentosa (RP), Stargardt's Disease, Leber hereditary optic neuropathy (LHON), Leber congenital amaurosis (LCA), Best disease, adult vitelliform macular dystrophy (AVMD), X-linked retinoschisis, blindness, a color vision disorder, color blindness, blue cone monochromacy, achromatopsia, incomplete achromatopsia, protan defects, deutan defects, tritan defects, disorders of the central macula, dry age-related macular degeneration (dry AMD), wet age-related macular degeneration (wet AMD), geographic atrophy, retinal telangiectasia, macular telangiectasia, Coats' disease, diabetic retinopathy, retinal vein occlusions, retinal ischemia, Familial Exudative Vitreoretinopathy (FEVR), glaucoma, Sorsby's fundus dystrophy, and combinations thereof.

13. A method of treating a cone photoreceptor cell-associated disorder in a subject in need of such therapy, comprising:
administering to the eye of the subject a recombinant vector comprising:
(a) a coding sequence encoding a human synoviolin 1 (SYVN1) protein or an orthologue, wherein the coding sequence has at least 90% identity to SEQ ID NO:1; and
(b) a promoter region, wherein the promoter region is specific for cone photoreceptor cells;
wherein the coding sequence is operatively linked to the promoter region; and
wherein in vivo expression of the SYVN1 protein in the cone photoreceptor cells of the subject serves to treat the cone photoreceptor cell-associated disorder in the subject.

14. The method of claim 13, wherein the recombinant vector further comprises an enhancer element upstream of the promoter, wherein the coding sequence is operatively linked to the enhancer element.

15. The method of claim 13, wherein the human SYVN1 protein comprises the amino acid sequence of SEQ ID NO: 1.

16. The method of claim 13, wherein the coding sequence is at least 90% identical to SEQ ID NO: 2.

17. The method of claim 13, wherein the cone photoreceptor cell-associated disorder is selected from the group consisting of rod-cone dystrophy, cone-rod dystrophy, progressive cone dystrophy, retinitis pigmentosa (RP), Stargardt's Disease, Leber hereditary optic neuropathy (LHON), Leber congenital amaurosis (LCA), Best disease, adult vitelliform macular dystrophy (AVMD), X-linked retinoschisis, blindness, a color vision disorder, color blindness, blue cone monochromacy, achromatopsia, incomplete achromatopsia, protan defects, deutan defects, tritan defects, disorders of the central macula, dry age-related macular degeneration (dry AMD), wet age-related macular degeneration (wet AMD), geographic atrophy, retinal telangiectasia, macular telangiectasia, Coats' disease, diabetic retinopathy, retinal vein occlusions, retinal ischemia, Familial Exudative Vitreoretinopathy (FEVR), glaucoma, Sorsby's fundus dystrophy, and combinations thereof.
